SEQUENCE LISTING (4 sequences)

SEQ ID NO: 1 - protein, 142 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 2 - protein, 120 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNK

SEQ ID NO: 3 - protein, 119 aa, Homo sapiens
GAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNK

SEQ ID NO: 4 - DNA, 5392 bp, Artificial Sequence
  expression plasmid pAL37 comprising truncated survivin DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgggtgcccctacattgccgcctgcctggcagccgtttctcaaggaccaccgcatctctacattcaagaactggccgttcttggagggctgcgcctgcaccccggagcgcatggccgaggctggcttcatccactgcccgactgagaacgagccagacttggcccagtgtttcttctgcttcaaggagctggaaggctgggagccagatgacgacccgattgaggaacataaaaagcattcgtccggttgcgctttcctttctgtcaagaagcagtttgaagaattaacccttggtgaatttttgaaactggaccgcgaacgcgccaagaacaaaattgcaaaggaaaccaataataagtaataggcggccgcactcgagcaccaccaccaccaccaccaccactaattgattaatacctaggctgctaaacaaagcccgaaaggaagctgagttggctgctgccaccgctgagcaataactagcataaccccttggggcctctaaacgggtcttgaggggttttttgctgaaaggaggaactatatccggat

(19) United States
(12) Patent Application Publication
(10) Pub. No.: US 2016/0279213 A1
(43) Pub. Date: Sep. 29, 2016

(54) Survivin-Directed Cancer Vaccine Therapy

(71) Applicant: MERCK PATENT GMBH, Darmstadt (DE)

(72) Inventors: Simon Geissler, Bad Homburg (DE); Patrizia Boniforte, Darmstadt (DE); Joerg Plaschke, Bensheim (DE); Markus Weigandt, Mannheim (DE); Stefan Jaekel, Darmstadt (DE); Roland Kellner, Heppenheim (DE); Thomas Rysiok, Dudenhofen (DE); Dirk Mueller-Pompalla, Muehltal (DE); Kenneth Hance, Groton, MA (US)

(21) Appl. No.: 15/036,629

(22) PCT Filed: Dec. 16, 2014
(86) PCT No.: PCT/EP2014/003384

§ 371 Date: May 13, 2016

(30) Foreign Application Priority Data
Dec. 16, 2013 (EP) 13005831.6

(51) Int. Cl. A61K 39/00; A61K 39/39; A61K 45/06; C07K 14/47

(52) U.S. Cl. CPC A61K 39/0011; C07K 14/4747; A61K 39/39; A61K 45/06; A61K 2039/55511; A61K 2039/55555; A61K 2039/572

(57) ABSTRACT

The invention relates to a therapeutically effective truncated survivin and its therapeutic use as vaccine in a liposomal preparation, wherein the survivin fragment is preferably gluconoylated, and said liposomal preparation elicits in-vivo anti-tumor activity. The invention is directed, in more detail, to a cancer vaccine comprising a fragment of human survivin that is specifically effective in conjunction with an lipid adjuvant. The invention relates in more detail to a liposomal vaccine delivery system comprising an active truncated survivin molecule as tumor antigen and a chiral cationic lipid, for example R-DOTAP acting as adjuvant, which is part of the liposome preparation, wherein the liposomal drug delivery system is optimized with regard to its lipid and adjuvant components, physical or physicochemical parameters, and the final therapeutic efficacy of the released truncated survivin molecules. The invention is finally related to a method of increasing CD4⁺/CD8⁺ T-cell responses in-vivo, thus resulting in an increased anti-tumor activity by providing a liposome preparation comprising said truncated preferably gluconoylated survivin.

### FIELD OF THE INVENTION

The invention relates to a therapeutically effective truncated survivin and its therapeutic use as vaccine in a liposomal preparation, wherein the survivin fragment is preferably gluconoylated, and said liposomal preparation elicits in-vivo anti-tumor activity. The invention is directed, in more detail, to a cancer vaccine comprising a fragment of human survivin that is specifically effective in conjunction with a lipid adjuvant. The invention relates in more detail to a liposomal vaccine delivery system, comprising a truncated survivin molecule as tumor antigen and a chiral cationic lipid, for example R-DOTAP acting as adjuvant, which is part of the liposome preparation, wherein the liposomal drug delivery system is optimized with regard to its lipid and adjuvant components, physical or physicochemical parameters, and the final therapeutic efficacy of the released truncated survivin molecules. The invention is finally related to a method of increasing CD4⁺/CD8⁺ T-cell responses in-vivo, thus resulting in an increased in-vivo anti-tumor activity by providing a liposome preparation comprising said truncated preferably gluconoylated survivin.

### TECHNICAL BACKGROUND OF THE INVENTION

Apoptosis is a genetic program of cellular suicide, and inhibition of apoptosis has been suggested to be an important mechanism involved in cancer formation by extending the life span of cells favoring the accumulation of transforming mutations.

Survivin, a protein that inhibits cellular apoptosis and belongs to the family of inhibitors of apoptosis proteins (IAPs), was discovered and characterized by Altieri and co-workers in 1997 (see e.g., WO 98/22589; Ambrosini et al., Nature Medicine. 917-921, 1997; Adida et al., American Journal of Pathology, 152, 43-49, 1998; Cirino et al., J Clinical Investigations, 99, 2446-2451, 1997; Adida et al., The Lancet, 351, 882-883, 1998).

Survivin is a 16.5 kDa cytoplasmic protein containing a single BIR and a highly charged carboxy-terminal coiled region instead of a RING finger, which inhibits apoptosis induced by growth factor (IL-3) withdrawal when transferred in B cell precursors. Survivin is overexpressed in a broad range of solid tumors and hematological malignancies while exhibiting limited expression in terminally-differentiated adult tissues. Around 40 genes have been identified in the human transcriptome that were overexpressed in all cancers but not in adjacent normal tissues. Of these 40 genes, the survivin gene (BIRC5) was identified as the fourth most commonly overexpressed tumor associated antigen in human cancer (Velculescu et al., Nat Genet 23(4): 387-8, 1999).

Survivin is a 142 amino acid (aa) protein that is encoded by the Birc5 gene (a member of the inhibitor of apoptosis gene family). The amino acid sequence of human survivin is depicted by SEQ ID NO: 1

N-- MGAPTLPPAW QPFLKDHRIS TFKNWPFLEG CACTPERMAE
AGFIHCPTEN EPDLAQCFFC FKELEGWEPD DDPIEEHKKH
SSGCAFLSVK KQFEELTLGE FLKLDRERAK NKIAKETNNK
KKEFEETAKK VRRAIEQLAA MD --C.

The overexpression of survivin in most human cancers suggests a general role of apoptosis inhibition in tumor progression, a notion substantiated by the observation that in the case of colorectal and bladder cancer, as well as neuroblastoma, expression of survivin was associated with an unfavorable prognosis. In contrast, survivin is undetectable in normal adult tissues. These characteristics qualify survivin as a suitable TAA for both diagnostic and therapeutic purposes.

Survivin is highly over-expressed in most human cancers, including colorectal (67%), none small cell lung (96%), breast (90%), prostate (83%), renal cell carcinoma (79%), ovarian (87%), bladder (88%), endometrial cancer (83%), etc. Increased expression of survivin correlates with poor clinical outcome in all those tumors mentioned above.

The expression of the survivin protein in cancer is associated with a poor prognosis, advanced disease at diagnosis, higher grade, resistance to therapy, and higher rates of recurrence. Survivin has multiple functions in determining the balance between cell division and apoptosis. First, survivin is expressed in a cell-cycle dependent manner (G2-M checkpoint) and shown to interact with the mitotic spindle apparatus to promote cell cycle progression. Second, survivin inhibits apoptosis by interfering with caspase-9 processing and the activation of downstream effector caspases (e.g., caspase-3) resulting in inhibition of the mitochondrial apoptotic pathway. Third, during cellular stress responses, survivin interacts with the molecular chaperone heat shock protein 90 to promote cell survival. The degree of survivin overexpression in cancer as well as its intracellular role in promoting the survival and continued growth of cancer cells makes survivin an attractive target for immunotherapy. Moreover, survivin expression has been identified in proliferating endothelial cells in the tumor microenvironment presenting an additional target of therapeutic vaccination.

The interest in the concept of using vaccination in the treatment of cancer has grown with the increasing understanding of the role that the immune system is a major player in the biological behavior and development of cancer. The path to cancer vaccine development goes from the choice of the target antigen(s), the formulation, and the regimen of immunization to the assessment of its impact. In contrast to prophylactic vaccines, which generally aim at generating high levels of protective antibodies, therapeutic vaccines aim at eliciting potent T cell responses that can mediate the destruction of tumor cells, or at least stopping their growth. The first major decision down the therapeutic vaccine development path concerns the type of antigen to be used. Hundreds of T cell defined tumor antigens have been identified so far. The second decision concerns the choice of the vehicle to deliver the antigen to the immune system. A relatively large variety of possibilities exist including naked DNA, mRNA, recombinant protein, synthetic peptide, recombinant viral or bacterial vectors or dendritic cells loaded ex-vivo with antigen. The current invention is related to protein-based therapeutic cancer vaccines in general, and to survivin-directed cancer vaccines in detail.

The strategy of many therapeutic protein- or peptide-based cancer vaccines is to preferentially deliver a tumor antigen to the major histocompatibility complex (MHC) class I machinery of the adaptive immune system resulting in the subsequent induction of antigen-specific CD8⁺ T cells that have the ability to recognize and lyse tumor cells in an antigen-specific manner. This is achieved in peptide-based vaccines by restricting the antigenic repertoire to MHC class I peptides with the potential limitation of eliciting inadequate cytokine support from CD4⁺ helper T cells. During the last decade a considerable number of MHC class I/II peptides have been discovered derived from survivin (see e.g., U.S. Pat. No. 6,245,523; EP 2 092 938; and EP 2 359 841). These findings strongly suggest that survivin acts as a tumor rejection antigen precursor, TRAP, which is processed by cells into peptides having TRA functionality. However, respective survivin-derived 9- to 15-mer peptides are usually less immunogenic compared to full-length survivin.

One of the benefits to presenting full-length tumor antigen to the immune system in a therapeutic cancer vaccine is the potential to induce robust humoral as well as CD4⁺ and CD8⁺ T cell responses in the vaccinated host (Davis et al., PNAS 101(29): 10697-702, 2004). The therapeutic benefit of targeting both CD4⁺ and CD8⁺ T cell epitopes has been reported previously using a survivin peptide-based DC vaccine in a preclinical model of cerebral glioma (Ciesielski et al., Cancer Immunol Immunother 57 (12): 1827-35, 2008).

Peptides and recombinant proteins are often poorly immunogenic by themselves, hence the need to administer them in conjunction with adjuvants. The role of adjuvants is at least two-fold. On one hand, adjuvants deliver antigens to the immune system during a period of time long enough to allow for efficient priming of the T cell response. On the other hand, adjuvants trigger the activation and maturation of dendritic cells. As a consequence, DCs, loaded with antigen, migrate to the proximal lymph nodes and acquire the ability to optimally present antigens for initiation of de novo T cell responses. The former adjuvant function is fulfilled by agents, such as mineral oils for emulsion formation (incomplete Freund's adjuvant), liposomes or biodegradable microspheres. Relatively few adjuvants are currently available for human use. These include alum and MF59. However, a growing number of molecularly defined adjuvants are in clinical development and used in clinical trials of cancer vaccination. These include various synthetic or recombinant TLR ligands, mineral oils, such as montanides, saponins, and even liposomes.

Liposomal nanoparticles used for cancer vaccination and comprised of the cationic lipid and vaccine adjuvant, R,S DOTAP (1,2-dioleoyl-3-trimethylammonium propane), have been established as an effective delivery system for DNA, peptides, and protein. Other cationic lipids applied both as lipid component and adjuvant are DSTAP, DMTAP, DODAP, DDAB, R,S DOTMA, R-DOTMA, S-DOTMA, R,S DOEPC, R-DOEPC, and S-DOEPC. The use of DOTAP in the liposomal formulation confers a net positive surface charge that is reported to facilitate the interaction between the nanoparticle and the APC (Foged et al., Int J Pharm 298(2): 315-22, 2005). Further, DOTAP promotes dendritic cell maturation, thereby enhancing antigen cross-presentation, through a reactive oxygen species (ROS)-mediated mechanism (see Yan et al., J Control Release 130(1): 22-8. 2008).

Recently it was shown that liposomal preparations comprising chiral cationic lipids as lipid components and adjuvant, used in their separated enantiomer forms, such as R-DOTAP and S-DOTAP, are extraordinarily effective in augmenting the immune response of the antigen, and even in inducing, activating and directly modulating the immune response (see e.g., WO 2009/129227, and WO 2013/039989).

Liposomes are well-recognized drug delivery vehicles. They are microscopic, nanoparticle-sized, closed vesicles which enclose an internal aqueous space separated from the external medium by a bilayer membrane composed of phospholipids preferably identical to phospholipids segment that make up the cell membranes. The structure of a liposome highly resembles the basic structure of a cell. They are also relatively biocompatible. Liposome presents the potential to deliver drugs to desired target within the body and to reduce the systemic toxicity. Thus, there is growing interest that improved formulation and better targeting strategies will lead the way to better treatment. Liposomes can be widely classified on basis of size, morphology, composition, method of preparation and functions. Briefly, liposomes exist in two common varieties from size stand point of view: Small Unilamellar Vesicles (SUVs) and Large Unilamellar Vesicles (LUVs). SUVs have typically size range from 20 nm to 100 nm. Intermediate Unilamellar Vesicles (IUVs) having size range from 100 nm to 200 nm are considered among SUVs. SUVs are single-shelled vesicles preferably produced as a result of high-intensity ultrasonication. Hydrophilic drugs are entrapped in internal aqueous core whereas hydrophobic drugs get entrapped in the bilayer membrane.

As mentioned, liposomal formulations have demonstrated multiple benefits as drug delivery vehicles. However, they must be used to carry very potent drugs due to their low encapsulated load. Lipid-based vesicles pose several other challenges, such as instability in the bloodstream, poor solubility of many drugs in the lipid/surfactant solution, poor storage stability and a rapid, burst release of drug. Liposomal formulations are also associated with severe side effects due to their accumulation in skin tissue. Thus, as of 2012, only twelve drugs with liposomal delivery systems have been approved and five additional liposomal drugs were in clinical trials.

Therefore, there is a current and strong need to provide highly effective and stable liposomal antigen delivery systems, which release the antigen of choice that has to initiate a desired enhanced immune response in-vivo, which is boosted by the presence of the highly effective adjuvant.

Survivin is a favorable candidate for a cancer vaccine with the potential for durable tumor-specific immune responses and a favorable safety profile for the following reasons:
Low expression in terminally-differentiated normal tissue.
Highly expressed in a various cancer types.
Expression associated with advanced stage, grade, and resistance to therapy
Inhibitor of apoptosis.
Promotes cell division and migration/metastasis.
Promotes angiogenesis in the tumor microenvironment.
Involved in cellular stress response.

Hence, it is an object of the present invention to provide a liposomal preparation for survivin-directed cancer therapy, which is stable and sufficiently effective in-vivo at least in the presence of the strong adjuvant DOTAP or structurally similar chiral cationic lipids.

One of the problems to be solved must be seen by the inherent incompatibility of the antigen and the adjuvant with regard to their different permanent net charges (positive net charge of R-DOTAP, and negative net charge of survivin), that cause loss of stability. Another problem can be seen in insufficient in-vivo immunogenicity of survivin in respective preparations.

By the inventive selection of the lipid components of the liposomal preparation, the modification of the survivin structure/sequence, and optimization of numerous physicochemical parameters, such as particle size, antigen payload, concentration and contents of the lipid components and DOTAP, the problems could be solved.

### SUMMARY OF THE INVENTION

Attempts with full-length survivin in liposomal preparation showed that these particles elicited unsatisfying biophysical protein properties. Above all, a loss of stability of survivin could be observed when mixed with DOTAP, the preferred adjuvant according to the invention (FIG. 1, FIG. 2).

Surprisingly, it was found that by using a C-terminally truncated survivin the stability problem could be solved. Truncating the survivin C terminus stabilizes the protein in solution by removing a hydrophobic patch that induces precipitation of the protein in solution. The ability of the protein to be processed into peptides and presented to the immune system does not require biological activity on the part of the protein—only structural integrity prior to coming in contact with the protoesomal complexes of the antigen presenting cell. In one aspect of the invention, the C terminally truncated survivin is biologically still active and effective.

Therefore, it is an object of the invention to provide a C-terminally truncated form consisting of the 1-119, 1-120, 1-121, 1-122, 1-123, 1-124, 1-125, 1-126, 1-127, 1-128, 1-129, 1-130, 1-131, 1-132, 1-133 amino acid residues of full-length survivin (SEQ ID NO: 1), calculated from the N-terminus.

Because of bio-engineering strategies, the first methionine residue may be cleaved off in preferred embodiments of the invention providing the following truncated survivins: 2-119, 2-120, 2-121, 2-122, 2-123, 2-124, 2-125, 2-126, 2-127, 2-128, 2-129, 2-130, 2-131, 2-132, or 2-133 of SEQ ID NO: 1.

All these short track sequences of survivin specified above show improved bio-physical properties compared to full-length survivin, in a liposomal preparation comprising, for example DOTAP, preferably R-DOTAP as lipid adjuvant, while eliciting similar pharmacological properties, each as compared to full-length survivin represented by the 142 amino acid residue sequence (SEQ ID NO: 1).

Thus the invention relates to the following truncated survivin amino acid sequences:

MGAPTLPPAW QPFLKDHRIS TFKNWPFLEG CACTPERMAE
AGFIHCPTEN EPDLAQCFFC FKELEGWEPD DDPIEEHKKH
SSGCAFLSVK KQFEELTLGE FLKLDRERAK NKIAKETNNK

(SEQ ID NO: 2): The Gly2-K120 sequence track of this sequence is designated according to the invention as drug substance of the invention or “dC-Survivin Drug Substance”, and identical with the sequence track 1-119 of SEQ ID NO: 3.

GAPTLPPAW QPFLKDHRIS TFKNWPFLEG CACTPERMAE
AGFIHCPTEN EPDLAQCFFC FKELEGWEPD DDPIEEHKKH
SSGCAFLSVK KQFEELTLGE FLKLDRERAK NKIAKETNNK

(SEQ ID NO: 3): The Gly1-K119 sequence track of this sequence is designated according to the invention as drug substance of the invention or dC-Survivin and identical with the sequence track 2-120 of SEQ ID NO: 2.

The invention is preferably related to truncated survivin consisting of the first 120 N-terminal amino acid residues of full length survivin, wherein preferably the methionine residue at position 1 is deleted (SEQ ID NO: 2 and SEQ ID NO: 3).

It was further surprisingly found that truncated survivin as specified above which, however, is gluconoylated to a certain degree elicits significantly improved in-vivo pharmacological properties compared to truncated non-gluconoylated survivin.

Therefore, it is an object of the invention to provide a C-terminally truncated survivin consisting of the first 118-122 N-terminal amino acids of full-length human survivin represented by SEQ ID No. 1, or a sequence having a homology to said truncated survivin of >90%, preferably >91%, >92%, >93%, >94%, >95%, >96%, >97%, >98, or >99%, and eliciting the same or almost the same biological activity, wherein the truncated survivin is gluconoylated at one or more positions.

In one specific aspect, the invention relates to a C-terminally truncated survivin consisting of the first 118-133 N-terminal amino acids of full-length human survivin represented by SEQ ID NO: 1, or a sequence having a homology to said truncated survivin of >90%, wherein the truncated survivin is gluconylated at one or more positions. In a preferred aspect, said C-terminally truncated survivin elicits the same biological activity.

The invention is specifically related to a respective C-terminally truncated survivin consisting of the first 120 N-terminal amino acids of the full-length human survivin; said truncated survivin is represented by the amino acid sequence (SEQ ID NO: 2):

### MGAPTLPPAW QPFLKDHRIS TFKNWPFLEG CACTPERMAE AGFIHCPTEN EPDLAQCFFC

### FKELEGWEPD DDPIEEHKKH SSGCAFLSVK KQFEELTLGE FLKLDRERAK NKIAKETNNK,

wherein the truncated survivin is gluconoylated at one or more positions.

In a preferred embodiment of the invention, at least the glycine residue at position 2 of SEQ ID NO: 2, or the respective 118-122 amino acid residue sequences as depicted above, is gluconoylated.

The invention is specifically related to the respective truncated survivin, wherein the methionine residue at N-terminal position 1 is removed (usually after protein expression steps); said truncated survivin is represented by the amino acid sequence (SEQ ID NO: 3):

GAPTLPPAW QPFLKDHRIS TFKNWPFLEG CACTPERMAE
AGFIHCPTEN EPDLAQCFFC FKELEGWEPD DDPIEEHKKH
SSGCAFLSVK KQFEELTLGE FLKLDRERAK NKIAKETNNK

wherein the truncated survivin is gluconoylated at one or more positions.

In a preferred embodiment of the invention, at least the glycine residue at position 1 of SEQ ID NO: 3, or the respective 118-122 amino acid residue sequences, is gluconoylated.

It has been shown by the inventors that the favorable properties can be further improved if the gluconoylation is carried out at lysine residues of the truncated survivins according to the invention. Preferably, the lysine residues at positions 23 and 103 of SEQ ID NO: 2, and 22 and 103 of SEQ ID NO: 3 respectively, may be gluconoylated, preferably by means of glucono-1,5-lactone, although other gluconoylating agents can principally be used. Therefore, it is an object of the invention to provide a respective truncated gluconoylated survivin, wherein the gluconoylation is carried out additionally at lysine residues, preferably at lysine residues at position 23 and/or position 103 of SEQ ID NOs: 2, or at positions 22 and/or 102 of SEQ ID NO: 3.

It was surprisingly found that the in-vivo pharmacological data, especially with respect to activated CD4⁺ and/or CD8⁺ T cell responses caused by liposomal preparations according to the invention are especially favorable if not all truncated survivin molecules in a respective composition are gluconoylated. Specifically, 40% gluconoylation elicited the most balanced survivin-specific CD4⁺ and CD8⁺ T cell responses. Higher levels of gluconoylation preferentially engaged CD8⁺ T cell responses at the expense of CD4⁺ helper T cell support. Thus, it is an object of the current invention to provide a survivin composition comprising truncated survivin as specified above, and respective truncated non-gluconoylated survivin, wherein in said composition 10-80%, preferably 10-60%, more preferably 35-45%, most preferably approximately 40% of the truncated survivin molecules in said composition of truncated survivin are gluconoylated.

The gluconoylation of the truncated survivin according to the invention provides the following advantage:
Synthetic gluconoylation gives access to a broad modification range, allowing systematic evaluation of its pharmacological benefit.
The established protocol provides a basis to design an efficient and cost effective process.
Gluconoylation of (truncated) survivin increases the antigenicity and efficacy of the therapeutic truncated survivin of the invention as tumor vaccine.
Synthetic gluconoylation of truncated survivin according to the invention is considered as optimization, having the potential to work around the major disadvantages associated with host cell gluconoylation.

The survivin according to the invention may be glycosylated or non-glycosylated. Preferably, it is non-glycosylated and manufactured by bacterial systems, like E. coli.

As mentioned above, it was the primary goal of this invention to provide a liposomal preparation eliciting survivin or survivin-like activity in-vivo after vaccination, wherein the liposomal preparation is optimized with regard to different physical and pharmacological parameters and comprises the very effective immune stimulating agent and adjuvant DOTAP, preferably its chiral component R-DOTAP or similar chiral lipid adjuvants, such as R- or S-DOTMA or R,S DOEPC. Thus, it is an object of this invention to provide a liposomal preparation comprising:

(i) truncated, preferably gluconoylated survivin as specified above, such as the 120 amino acid residues containing sequence of SEQ ID NO: 2, or the 119 amino acid residues containing sequence of SEQ ID NO: 3, or respective modified sequences with a sequence homology of >90%, preferably >95%, or a survivin composition of respectively truncated survivin, wherein 10-60%, preferably 35-45% of the truncated survivin molecules are gluconoylated as described, and
(ii) at least one adjuvant, wherein the at least one adjuvant is a chiral cationic phospholipid acting as immunomodulator, preferably selected from the group consisting of R,S DOTAP, R-DOTAP, S-DOTAP, R,S DOTMA, R-DOTMA, S-DOTMA, R,S DOEPC, R-DOEPC, and S-DOEPC.

It was found by the inventors, that the above-specified chiral cationic adjuvant lipids are especially effective if applied in a concentration of 2-8 mM, preferably 3-5 mM, and most preferably approximately 4 mM, above all but not exclusively, in connection with R-DOTAP within the liposomal preparation. The contents of the lipid adjuvant within the liposomal preparation may vary, but interestingly, a content between 15 and 30% (mol/mol) elicits the best results with respect to pharmacological efficacy.

It was further shown by the inventors that—apart from the presence of the chiral cationic lipid—the quality and quantity of the lipid composition plays an important role with respect to the favorable properties of the liposomal preparation according to the invention.

Therefore, it is a further object of the invention to provide a liposomal preparation based on truncated survivin and at least one chiral cationic lipid adjuvant, such as DOTAP, as described above, which comprises cholesterol and at least one phospholipid selected from the group consisting of phosphatidylcholine (PC), phosphoethanolamine (PE), and phosphoglycerole (PG).

In a preferred embodiment of the invention, the phospholipid is a phosphatidylcholine (PC) selected from the group consisting of DLPC, DSPC, DPPC, DMPC, DOPC, EPC and EPC3. As shown below, egg phosphatidylcholine (EPC), which is a crude mixture of different PCs, is mostly preferred because it shows the most significant effect, for example, on CD8⁺ triggered response in-vivo compared to other phosphatidylcholines, such as DMPC. Nonetheless other known phospholipids can be used and tested for preparing the liposomal compositions according to the invention

The liposomal preparations according to the invention may comprise cholesterol in a content of 10-60% (mol/mol), preferably 20-45%, more preferably 30-45%, most preferably 45%, and a phosphatidylcholine (PC) as described above, of a content of 30-80% (mol/mol), preferably 30-50%, most preferably EPC of a content of approximately 35%.

A liposomal preparation according to the invention comprises nanoparticles of varying size between 40 nm and 500 nm. Preferred liposomal compositions comprise liposomal particles, wherein at least 70% of the particles, preferably at least 80% of the particles, have a size of 50-500 nm, preferably 100-300 nm, most preferably 150-250 nm, because these particles elicit the most favorable results with respect to physical and biological properties.

Furthermore, it was shown, that especially favorable results can be obtained when the particle surface charge is 17 mV dependent on the antigen payload, which according to the invention, is 0.5-1.5 mg/mL, preferably 0.75-1.0 mg/mL. A positive surface charge (zeta potential) enhances the immune and antitumor activity of DOTAP-containing liposomal nanoparticles.

From the above it can be seen that all relevant parameters and components of the liposomal nanoparticles have been varied resulting in an optimized liposomal preparation which elicit partially surprising properties, and is designated according to the invention as drug product of the invention, or “dc-Survivin Drug Product”.

Therefore it is a specific object of the invention to provide a respective liposomal preparation comprising:

(i) truncated (gluconoylated) survivin and compositions of truncated survivin as specified above and in the claims, preferably the sequence of SEQ ID NO: 2 or 3,
(ii) the lipid components R-DOTAP, cholesterol and EPC,
(iii) the R-DOTAP concentration in said preparation is 3-5 mM,
(iv) liposomal particles, wherein at least 70% have a particle size of 150-250 nm, and
(v) an antigen payload of 0.5 mg/mL (±0.1 mg/mL).

In a preferred embodiment the drug product is characterized by the following parameters:

(i) the truncated gluconoylated, non-glycosylated survivin represented by SEQ ID NO: 3, wherein approximately 40% (±2-5%, preferably ±5%) of said truncated survivin is gluconoylated, and
(ii) the contents of the lipid composition is: 10-30%, preferably approximately 20% (±1%) R-DOTAP; 25-55%, preferably approximately 45% (±1%) cholesterol; and 30-60%, preferably approximately 35% (±1%) EPC (mol/mol).

The liposomal particles, preferably the phospholipid components of the liposomal preparations according to the inventions may be pegylated according to standard methods. These pegylated lipid particles of the invention show enhanced stability and a strong tendency with respect to reduced sensitivity of the formulation toward lowered salt concentrations, thereby influencing and improving significantly protein or formulation precipitation. However, the pegylated liposomal preparations according to the invention elicit surprisingly a lower in-vivo efficacy (CD4⁺/CD8⁺ T cell responses and anti-tumor activity) as compared to non-pegylated particles. However, pegylation of the lipid particles may be used according to the invention in order to modulate the immune response in the patient if needed, especially in individuals with overreacting immune responses. Therefore, it is an object of the invention to provide a liposomal preparation or vaccine composition as specified above and below, wherein the liposomal particles are pegylated, preferably between 0.5% and 5% (mol/mol total lipid content).

The invention is related further to liposomal and vaccine preparations according to the invention, which are lyophilized for storage reasons. The presence of sucrose in low concentration between 2.0 and 2.5% (w/v) stabilizes the preparations sufficiently without compromising the pharmacological efficacy of the lyophilized preparation. The preparations according to the invention may also comprise antioxidants, preferably monothioglycerole (MTG), for preservation from oxidative damage.

The liposomal and vaccine preparations as described above and below are used according to the invention for preparing a vaccine composition, which is intended for vaccination treatment.

Thus, it is an object of the present invention to provide an immune stimulatory vaccine comprised of the liposomal preparation as specified above and below, which may further comprise carriers, excipients, additives, diluents, additional immune enhancers or immune effector molecules.

The vaccine compositions according to the invention may be combined together or separately (concurrent or sequential administration) with other therapeutically effective drugs, such as anti-tumor agents and/or other therapy forms, such as radiation.

In a preferred embodiment, the liposomal vaccine preparation is combined with chemotherapeutic agents, such as cyclophosphamide, carboplatin, or paclitaxel, or with anti-tumor antibodies or antibody-cytokine fusion proteins, such as NHS-IL12, as described below. These combinations show enhanced in-vivo efficacy (FIG. 21-24). Therefore, in more detail, it is a further object of the invention to provide a pharmaceutical composition comprising a liposomal preparation or an immunostimulatory vaccine composition, in each case as described herein, together with an anti-tumor agent selected from the group consisting of cyclophosphamide, carboplatin, paclitaxel and NHS-IL12. In one preferred aspect, the invention relates to said pharmaceutical composition for use in the prophylaxis or treatment of a human individual, wherein the individual suffers from cancer or a cancer-related disease.

The invention is finally related to the immunostimulatory vaccine or liposomal preparation as described for use for the prophylaxis or the treatment of a human individual by vaccination, wherein the individual suffers from cancer or a cancer-related disease or has a predisposition for it. The invention is especially related to respective methods of treating patients which suffer from cancer or have a preposition for it by vaccination, wherein the vaccination triggers survivin-specific immune responses in said individual, for example by modulating, preferably enhancing CD4⁺ and/or CD8⁺ T-cell responses by the patient's challenged immune system, and results in inhibiting or preventing tumor growth in the patient.

According to the invention, the following parameters of the vaccine were optimized for the formulation and enhancement of the immune response compared to full-length survivin:
Survivin Protein Modification: controlled synthetic gluconoylation between 10 and 80%, preferably 10-60% affects optimal induction of survivin-specific CD4⁺ and CD8⁺ T cell responses.
R-DOTAP Concentration: 3.5-4.5 mM DOTAP include an optimal balance of survivin-specific CD4⁺ and CD8⁺ T cell responses.
Particle Surface Charge: a zeta potential of >17 mV, preferably around 18 mV, induces an optimal balance of survivin-specific CD4⁺ and CD8⁺ T cell responses.
Particle Size: larger nanoparticles, preferably >200 nm, enhance survivin-specific CD8⁺ T cell IFN-gamma production.
Lipid Composition: EPC helper lipids consistently outperform synthetic helper lipids, such as DMPC, etc. in developing a cellular immune response.
Excipients for Cryopreservation: 2-3% sucrose supports stabile and active lyophilized vaccine preparations.

The formulations of the inventions enhance aqueous stability of truncated survivin and overcome inherent incompatibility of R-DOTAP and full-length survivin by switching to truncated survivin as specified. Pharmacological activity was maintained versus full-length native survivin and increased compared to truncated non-gluconoylated survivin by applying truncated gluconoylated survivin as described (see FIG. 2-FIG. 14).

### SHORT DESCRIPTION OF THE FIGURES ACCORDING TO THE INVENTION

FIG. 1A: DOTAP liposomes at 8 mM lipid concentration with increasing amount of survivin. From left to right: 0 μg/mL protein, 41 μg/mL protein, 3.1 mg/mL protein. Precipitation of a DOTAP-protein complex is induced with increasing protein concentration

FIG. 2B: Stability assessment of full length versus C-terminal truncated survivin in a matrix of pH and salt conditions. Green indicates favorable, red unfavorable conditions. A relevant extension of protein compatible conditions towards low salt and lower pH is being observed for survivin of 1-120 amino acids.

FIG. 2: Titration of lipid composition and influence of particle size distribution in 96w format for ternary mixtures containing either DOTAP/CHO/DMPC or DOTAP/CHO/DPPC, samples were measured at t0 and after 1 week storage at 2-8° C. Values are obtained after pooling and measuring three preparations.

FIG. 3: Titration of liposome composition regarding different PC combinations in 96w format: DMPC+DPPC, DOPC+DPPC, DOPC+DSPC, appropriate particle size distributions are obtained when using combinations of DOPC+DPPC or DPPC+DMPC. Values represent the pool of three vials each.

FIG. 4: Titration of liposome composition in 96w format regarding different ratios of DPPC and DOPC at fixed contents of DOTAP (20% mol/mol) and cholesterol (45% mol/mol), appropriate particle size distributions are obtained in a wide range of ratios. Values represent the pool of three vials each.

FIG. 5: Titration of liposome composition regarding different PC combinations in 96w format at reduced salt content of 100 mM KCl. DMPC proves to be superior in comparison to DPPC and DSPC to stabilize surviving-loaded liposomes. Reduction of DOTAP content also demonstrates to stabilize the liposome. Values represent the pool of three wells each.

FIG. 6: Titration of liposome composition in 96w format in presence of reduced KCl content (50 mM). Different ratios of DSPE-PEG2000 (1-5% mol/mol) and different helper lipids were investigated regarding formulation feasibility and short term stability over 1 week at 4° C. DMPC, DPPC and DOPC were applied as phospholipids. Values represent the pool of three wells each.

FIG. 7: Influence of concentration of adjuvant. CD4 and CD8 readout after 2 vaccinations in C57BL/6 mice, Vaccine composition: 100 nm DOTAP liposomes, 0.5 mg/mL antigen payload, lipids: 20% DOTAP, 35% EPC, 45% cholesterol.

A: T-cell memory responses insinuate a proliferation maximum at around 2 mM DOTAP.

B: In contrast, the highest frequency of SVN-specific IFN-γ producing CD8⁺ T cells was observed at the highest concentration of DOTAP in the formulation.

Control: Non-specific immune activation was quantified by stimulating isolated CD8⁺ T cells from vaccinated mice with the MHC class I restricted ovalbumin peptide (SIINFEKL).

FIG. 8: Influence of DOTAP concentration—2 mM versus 4 mM R-DOTAP.

A: CD4⁺ T cell proliferation, and

B: CD8⁺ IFN-γ production after 2 vaccinations in C57BL/6 mice, Vaccine composition: 100 nm DOTAP liposomes, 0.5 mg/mL antigen payload, lipids: 20% DOTAP, 35% EPC, 45% cholesterol.

Control: Non-specific immune activation was quantified by stimulating isolated CD8⁺ T cells from vaccinated mice with the MHC class I restricted ovalbumin peptide (SIINFEKL). 4 mM DOTAP induced an optimal balance of survivin-specific CD4⁺ and CD8⁺ T cell responses.

FIG. 9: Effect of zeta potential and vaccine payload on survivin-specific CD4⁺ and CD8⁺ T cell responses. CD4 and CD8 readout after 2 vaccinations in C57BL/6 mice, Vaccine composition: 10 mM lipid concentration, lipids: 20% DOTAP, 35% EPC, 45% cholesterol.

A: SVN-specific CD4⁺ T cell proliferation as measured by ³H-thymidine uptake in isolated CD4⁺ T cells stimulated with antigen presenting cells pulsed with purified full-length survivin protein.

B: SVN-specific CD8⁺ T cell IFN-γ production as measured by ELISPOT assay using isolated CD8⁺ T cells stimulated with antigen presenting cells pulsed with MHC class I restricted SVN peptides.

Particle Surface Charge: −6 mV to +33 mV. Vaccine payload leads to a change in particle surface charge. Zeta potential of +18.3 mV induces an optimal balance of survivin-specific CD4⁺ and CD8⁺ T cell responses.

FIG. 10: Influence of particle size on cytotoxic immune responses. The formulation consisted of 20% R-DOTAP/35% EPC/45% cholesterol (mol/mol) at a total lipid concentration of 10 mM, drug load 0.5 mg/mL each. Larger nanoparticles (200 nm) enhanced survivin-specific CD8⁺ T cell IFN-γ production. EPC helper lipids consistently outperformed synthetic helper lipids (e.g., DMPC) in developing a cellular immune response.

FIG. 11: Comparison of DMPC versus EPC as phospholipid component. Liposomes consisted of 20% DOTAP/25% EPC/45% cholesterol (mol/mol) or 20% DOTAP/50% DMPC/30% cholesterol, lipid concentration: 10 mM, drug load: 0.5 mg/mL, size: 105 nm (EPC) versus 129 nm (DMPC), data acquired after two weekly vaccinations, N=10 mice per group.

FIG. 12: Cytotoxic immune responses of Pegylated liposomes loaded with:

A: Two vaccinations.

B: Four vaccinations, lipid concentration: 10 mM, size: 50-60 nm, antigen payload: 0.5 mg/mL, n=10 mice per group.

3 and 5% pegylation.

FIG. 13: Comparison of pegylated survivin liposomes (3%, 5%) versus a non-pegylated formulation (0%), liposome composition: 20 mM lipid concentration, app. 200 nm size, antigen load 0.5 mg/mL, data demonstrate superior cytotoxic effect of the non-pegylated formulation.

FIG. 14: Comparison of liquid (EPC liquid) and lyophilized (EPC lyo) liposomes after reconstitution, liposome composition: 20 mM, 20% DOTAP/35% EPC/45% cholesterol, size: 200 nm, antigen load: 0.5 mg/mL, EPC lyo additionally contains 100 mg/mL sucrose.

FIG. 15A: Gluconoylation scheme.

FIG. 15B: Survivin gluconoylation range achieved by utilization of glucono-1,5-lactone reagent.

FIG. 15C: Evaluation of reaction parameters and their effect on survivin gluconoylation.

FIG. 16: Effect of survivin gluconoylation on survivin-specific CD4⁺ and CD8⁺ T cell responses.

FIG. 17: Effect of survivin gluconoylation on dC-Survivin's antitumor efficacy in a subcutaneous MC38/SVN tumor model.

FIG. 18: Synthetic gene of 1-120 aa truncated survivin according to the invention. The expression plasmid AL37 comprises the DNA sequence coding for the truncated survivin (SEQ ID NO: 2; 1-120 aa). After expression the truncated survivin of SEQ ID NO: 3 (corresponds to 2-120 of SEQ ID NO: 2, omitting the first methionine residue) was released into the medium.

FIG. 19: Restriction sites in pAL37.

FIG. 20: DNA sequence and features on expression plasmid pAL37 (SEQ ID NO: 4) comprising truncated survivin DNA coding for SEQ ID NO: 2/3.

FIG. 21: Combination of truncated survivin according to the invention (2-120 aa of SEQ ID NO: 3) with cyclophosphamide (CPA).

FIG. 22: Combination of truncated survivin according to the invention (2-120 aa of SEQ ID NO: 3) with antibody-cytokine fusion protein NHS-IL12.

FIG. 23: Combination of truncated survivin according to the invention (2-120 aa of SEQ ID NO: 3) with paclitaxel in SVN.Tg mice with ovarian tumors.

FIG. 24: Combination of truncated survivin according to the invention (2-120 aa of SEQ ID NO: 3) with carboplatin in SVN.Tg mice with ovarian tumors.

### DETAILED DESCRIPTION OF THE INVENTION

According to the definition by this invention the term “drug substance” refers to “dC-Survivin” (2-120 aa dimeric survivin construct truncated at C-terminus), shortly designated as truncated survivin of the invention based on SEQ ID NO: 3, wherein the truncated survivin is specifically gluconoylated at least at Gly1 of SEQ ID NO: 3.

According to the definition by this invention the term “drug product” refers to the liposomal preparation comprising the “drug substance” as specified above, and optimized as described above and below. In more detail the “drug product” according to the invention is: (i) the truncated survivin represented by SEQ ID NO: 3 and non-glycosylated, wherein 40% (±0.5%) of said truncated survivin is gluconoylated, and (ii) the contents of the lipid composition is: 10-30%, preferably approximately 20% (±1%) R-DOTAP; 25-55%, preferably approximately 45% (±1%) cholesterol; and 30-60%, preferably approximately 35% (±1%) EPC (mol/mol).

### Choice of Expression System

The small size and the intention to generate the polypeptide void of post-translational modifications made truncated survivin a particular suited candidate for production in a prokaryotic expression system, such as E. coli. Due to its natural cytoplasmic localization, survivin is adapted to a reducing environment. In course of development it became evident, that the survivin protein needs continuous anti-oxidative protection to maintain structural integrity. This requirement clearly favored an intracellular targeting for recombinant production. In addition, a feasibility study in a Pichia system revealed ineffective secretion properties of survivin. These considerations made E. coli the choice of production host, while maintaining the protein product intracellular. It was further decided to utilize an inducible promoter system (laclq/T7) in combination with an episomal vector. A plasmid based system has the advantage of good accessibility for modifications to the expression cassette in course of project progression. Controlling expression by induction allows tuning and optimization of transcript levels. It further avoids negative selection pressure in the cell banking process and during growth phase of fermentation cultures.

Mass spectrometry analysis of the E. coli produced survivin revealed two species with a mass increase of +178 and +258 Da relative to the main form. This pattern is indicative for a protein modification by a reactive sugar derivative, creating the so called phospho-gluconoylation. It arises from a spontaneous reaction of primary amino groups in proteins with phospho-gluconolactone, a metabolic intermediate at the interface of glycolysis and pentose phosphate pathway. After the initial phospho-gluconoyl protein adduct (+258 Da) is formed, there is a partial hydrolysis to the gluconoyl form (+178 Da) (see Geoghegan et al., Anal Biochem 267(1): 169-84, 1999). In the case of survivin, this reaction was confined to the alpha-amino group at Gly2. Under the range of expression conditions tested, up to 12% of the survivin protein was modified based on this mechanism. The modification was unintended and evaluated as a risk regarding manufacturing control. It was further seen to potentially raise regulatory concern. However, intense efforts to either remove the modified species in the purification process, or to avoid their generation by appropriate fermentation settings, were unsuccessful.

Uncontrolled phospho-gluconoylation occurs as the host cell accumulates the metabolite phospho-gluconolactone. Therefore, it was a goal of the current invention to provide an E. coli strain, wherein no gluconoylation takes place, although it was surprisingly found by these investigations that gluconoylated truncated survivin according to the invention is pharmacologically more active compared to non-gluconoylated survivin.

Thus, the inventors developed an expression host that does not natively (phospho)-gluconoylate the expressed truncated survivin. Instead, (phospho)-gluconoylation should be carried out after expression by synthetic means and in a controlled manner. However, it should be noted that truncated gluconoylated survivin, wherein gluconoylation is obtained by a natural or bioengineered expression host system is included in the invention.

A suitable and preferred E. coli strain which can be used according to the invention for expression of the truncated survivin of the invention is E. coli BL21(DE3) (Novagen). However, this strain naturally suffers from insufficient lactonase activity and hydrolyzes this reactive intermediate into the inert gluconoic acid. This purchasable strain was bioengineered according to the invention by introducing a copy of a (phospho)-gluconolactonase gene into the genome of this expression strain. This strategy completely abolished the occurrence of phospho-gluconoylated and gluconoylated survivin species, and opens the way to introduce gluconoylation in a controlled manner by synthetic means principally known in the art. In principal, also other bacterial strains with an insufficient lactonase activity, can be engineered accordingly, and thus can be used to produce the truncated survivin of the invention.

The strain resulting from bioengineering an E. coli strain as described above, preferably E. coli BL21(DE3), is taken according to the invention for transformation with the expression plasmid containing the truncated survivin of the invention. The specific strain according to the invention is designated E. coli T7E2(#1).

For producing the non-glycosylated and originally non-gluconoylated truncated survivin according to the invention, a plasmid was constructed by which E. coli T7E2 was transformed. The plasmid is designated pAL37 and the sequence and features are depicted in FIGS. 19 and 20. Of course, variants and modifications of the plasmid construct are principally included in the invention, especially with leading sequences, promoter sequences, restriction sites, antibiotic resistance, etc. It should be pointed out that the use of (synthetically) glycosylated truncated survivin (in order to modify the biological and physical properties of survivin) is covered by the gist of the invention as well.

### Modification Reaction (Gluconovlation)

The truncated survivin according to the invention is preferably non-gluconoylated when expressed by the bacterial expression host. In order to achieve a controlled and desired gluconoylation rate of the bioengineered truncated survivin, the introduction of gluconoyl residues is carried out synthetically by standard methods. The gluconoylation is achieved by reacting free amino groups with a standard gluconoylating reagent, such as glucono-1,5-lactone (FIG. 15A). Possible free amino residues within the truncated survivin of the invention are glycine at position 2 or SEQ ID NO: 2 after cleaving off the methionine residue, and glycine at position 1 of SEQ ID NO: 3. Further, candidates for gluconoylation are all lysine residues in the truncated molecule, although lysine 23 and lysine 103 (SEQ ID NO 2) or lysine 22 and lysine 102 (SEQ ID NO: 3) are preferred lysine residues, because gluconoylation at these positions elicit a survivin with best pharmacological properties.

According to the invention the vaccine with the gluconoylated truncated survivin of the invention triggers a stronger immune response in-vivo than the respective vaccine with the same but non-modified survivin.

Host cell mediated gluconoylation of survivin originated as an unintended consequence of a suboptimal metabolic situation in the employed E. coli strain, such as BL21(DE3). The low achievable degree of modification (up to 12%) and the limited control over this event were seen detrimental to capitalize on this mechanism. To enable a systematic assessment of the potential pharmacological benefit of survivin gluconoylation at a broader modification range, a synthetic approach has been designed instead. Non-modified survivin DS bulk, originating from the expression strain T7E2#1, is reacted with glucono-1,5-lactone. The degree of modification increases with the amount of glucono-1,5-lactone offered. Modification levels up to 80%, preferably 65%-80%, were achieved (FIG. 15B).

The reaction remains remarkably selective for the alpha-amino group at Gly2 (SEQ ID NO: 2), or Gly1 (SEQ ID NO: 3), likely due to its unique pKa properties. However, at elevated lactone concentrations modification of two lysine residues at low level has been observed. Two methods with orthogonal detection principles are available to quantify gluconoylation levels of survivin (cIEF by charge, ESI-MS by mass, RP-HPLC by stationary phase interaction properties). Due to the spontaneous hydrolysis of the lactone in aqueous solution, it has to be added as powder and in significant molar excess over the protein component. Impact of key reaction parameters like temperature, reaction time, and pH were evaluated in addition to amount of gluconoylation reagent (FIG. 15C).

### Formulation Feasibility of the Buffer System Used, Antioxidants Included

In the framework of the development of the truncated survivin of the invention, adaptations to the buffer medium were made. One suitable formulation buffer with optimized characteristics comprises of 50 mM Na-P, 150 mM NaCl, 1 mM DTT, pH 7.5. As DTT is not approved by health authorities the buffer composition needed to be reworked regarding accepted antioxidants. Furthermore type of buffer salt system, pH and monovalent salt concentration were explored to define the optimum stability range of the survivin construct. Results can be summarized as follows:

Ethanol and t-butanol were tested in a range from 5-20%. Increasing oxidative dimerization is observed with increasing solvent concentration after 5d at 25° C. Least detrimental effects were detected with 5% t-butanol.

Truncated survivin exhibits a pl at 6.1. When performing a pH titration in a range from 4 to 9, a stability minimum was detected at 5.0<pH<6.0 and precipitation occurred which corresponds to the net molecule charge at these pH values. Remarkably, chemical stability (oxidation) was reduced at higher pH values of ≧7.0 when testing without addition of antioxidants. pH stability at pH 6 could be dramatically enhanced via introducing 150 mM NaCl into the formulation. At optimum salt concentration the API can be stabilized within a pH range of 6-8 with a stability optimum at pH 6-6-5. NaCl demonstrated to stabilize dC-Survivin at pH values near the pl of the molecule. It furthermore proved to be a vital component during processing of drug loaded R-DOTAP containing liposomes. Without or with an insufficient NaCl concentration (<150 mM) the protein precipitates in the presence of the cationic liposomes.

The final preferred buffer composition was defined to consist of the following excipients: 20 mM K₂HPO₄, 150 mM KCl, 10 mM MTG, pH 7, however modifications in composition and content are also covered by the invention.

The following antioxidants were tested with regard to preservation of truncated survivin according to the invention from oxidative damage: ascorbic acid, K-sulfite, Na-sulfite, Na-thiosulfate, cysteine, methionine, monothioglycerole, Na-formaldehyde sulfoxate, and propyl gallate. All these excipients are used in marketed parenteral products according to the FDA ingredient list. Antioxidative performance was tested after incubation for 5d at 25° C. Results demonstrated the superiority of monothioglycerole (MTG) in comparison to other antioxidants. Only MTG, methionine and Na-thiosulfate prevented oxidation in the test protocol. Propyl gallate and ascorbic acid caused unwanted reactions and lead to a discoloring of the test samples. The concentration of MTG was titrated from 0-10 mM in 20 mM K₂HPO₄, 150 mM KCl, pH 7.0. Prevention of oxidation (5d at 25° C.) was dependant on the MTG concentration.

### Lipids and Phospholipids Used in the Liposomal Preparation of the Invention

There are a numerous lipid compounds and derivatives which can be used for preparing the lipid components of the liposomal particles according to the invention. The liposomal preparations usually comprise lipid compounds, such as cholesterol or alpha-tocopherol, cationic phospholipids and neutral phospholipids. The cationic component is necessary according to the invention to compensate the negative charge of the survivin fragment used in the liposomal preparations of the invention.

(A) Chiral Cationic Phospholipids

The cationic phospholipid components are preferably of chiral nature because the separated enantiomeric forms show increased biological and pharmaceutical efficacy. They may be preferably selected from the group consisting of R,S DOTAP, R-DOTAP, S-DOTAP, R,S DOTMA, R-DOTMA, S-DOTMA, R,S DOEPC, R-DOEPC, and S-DOEPC, but are not necessarily limited to these agents.

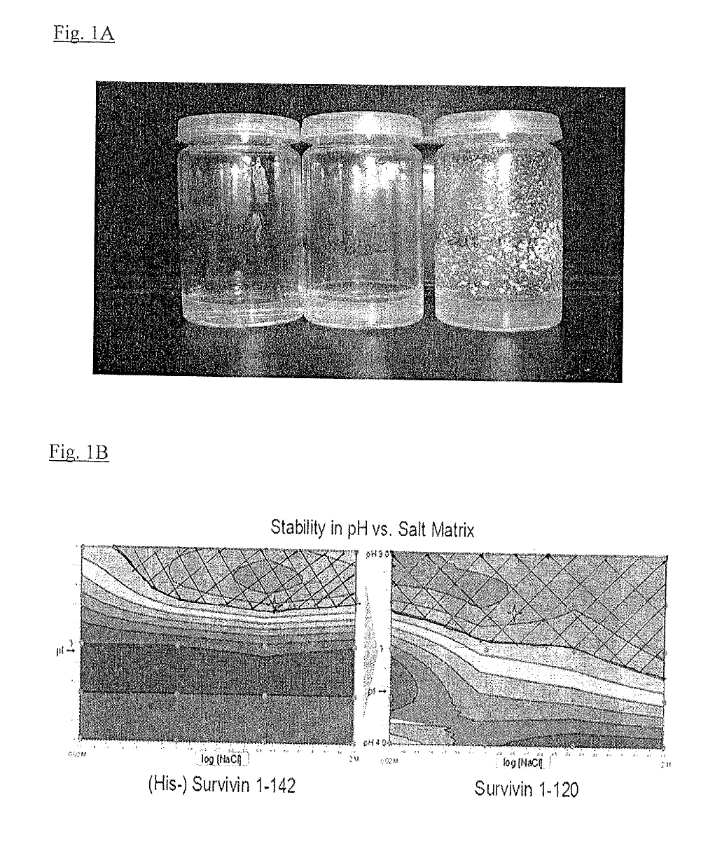

Formulation feasibility of truncated survivin liposomes is guided by the inherent incompatibility of truncated survivin and the adjuvant component, preferably R-DOTAP. FIG. 1 is giving an impression of this phenomenon which is due to uncontrolled interaction between the permanent positive charge of DOTAP and the net negative charge of survivin at physiologic conditions. In the course of formulation development one major goal was to suppress undesired electrostatic interactions while maximizing the DOTAP content per liposome. DOTAP is not only acting as lipid component as part of the liposomal bilayer but acts strongly as adjuvant in vaccination approaches and settings (here in context with truncated survivin).

By titrating DOTAP in a range of 0-50% (mol/mol) it could be demonstrated that charge interactions can be controlled in a range of 0-30% (mol/mol) DOTAP. Further it could be shown that mixtures of DOTAP and one neutral lipid component did not result in particles in the envisaged target size. Preferred results were obtained when combining DOTAP, cholesterol and one PC at distinct ratios, for example: 10-30%, preferably approximately 20% (±1%) R-DOTAP; 25-55%, preferably approximately 45% (±1%) cholesterol; and 30-60%, preferably approximately 35% (±1%) EPC (mol/mol).

(B) Other Lipids and Phospholipids

It was found that a control of the charge interactions can be achieved via introducing neutral lipid components (phosphadityl cholines (PC), including Egg PC (EPC), cholesterol, and tocopherol) into the liposome bilayer. Examples of suitable neutral phospholipids are: phosphatidic acid (phosphatidate) (PA), phosphatidylethanolamine (cephalin) (PE), phosphatidylcholine (lecithin) (PC), phosphatidylserine (PS), phosphatidylinositol (PI), phosphatidylinositol phosphate (PIP), phosphatidylinositol bisphosphate (PIP2), and phosphatidylinositol triphosphate (PIP3).

Furthermore synthetic phospholipid derivatives can be used according to the invention:
Phosphatidic acid (DMPA, DPPA, DSPA)
Phosphatidylcholine (DHPC, DLPC, DMPC, DPPC, DSPC, DOPC, POPC, DEPC)
Phosphatidylglycerol (DMPG, DPPG, DSPG, POPG)
Phosphatidylethanolamine (DMPE, DPPE, DSPE DOPE)
Phosphatidylserine (DOPS)

Table 1 is giving an overview of the lipids preferably used according to the invention for preparing the liposomal preparations of the invention.

| Cationic phospholipid | Neutral (helper) phospholipids | | Lipids |
|---|---|---|---|
| R-DOTAP | PC: | DLPC, DMPC, DPPC, | Cholesterol |
| (enantiomeric DOTAP) | | DSPC, DOPC, EPC, | (CHO); alpha- |
| | | EPC3 | tocopherol |
| | PE: | DMPE, DPPE, DSPE, | |
| | | DSPE-PEG2K, | |
| | | DPPE-PEG5K | |
| | PG: | DMPG, DPPG, DSPG | |

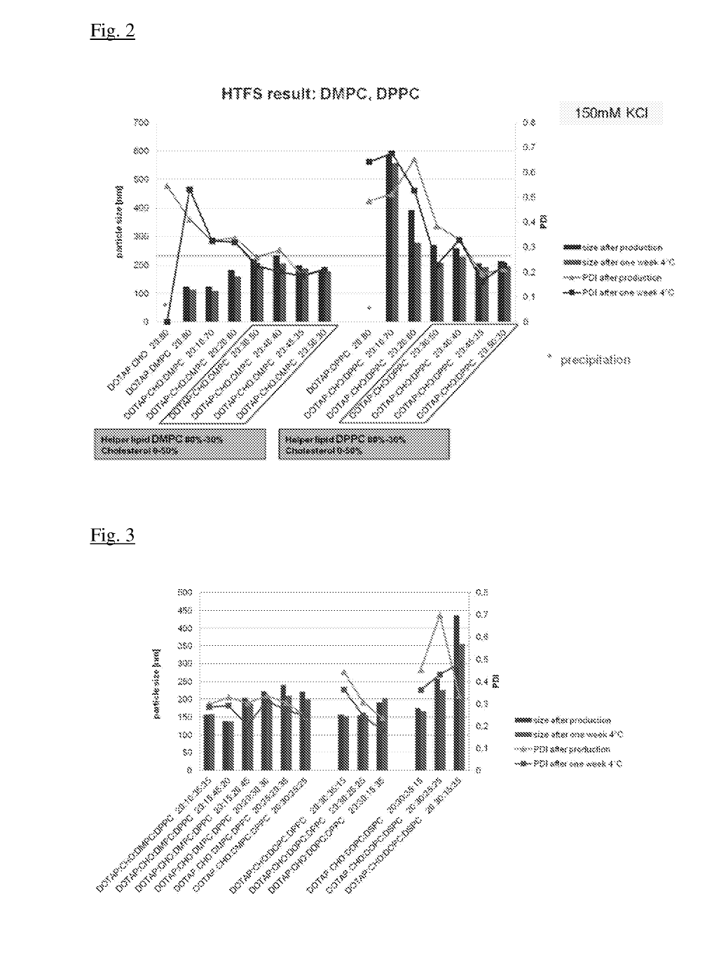

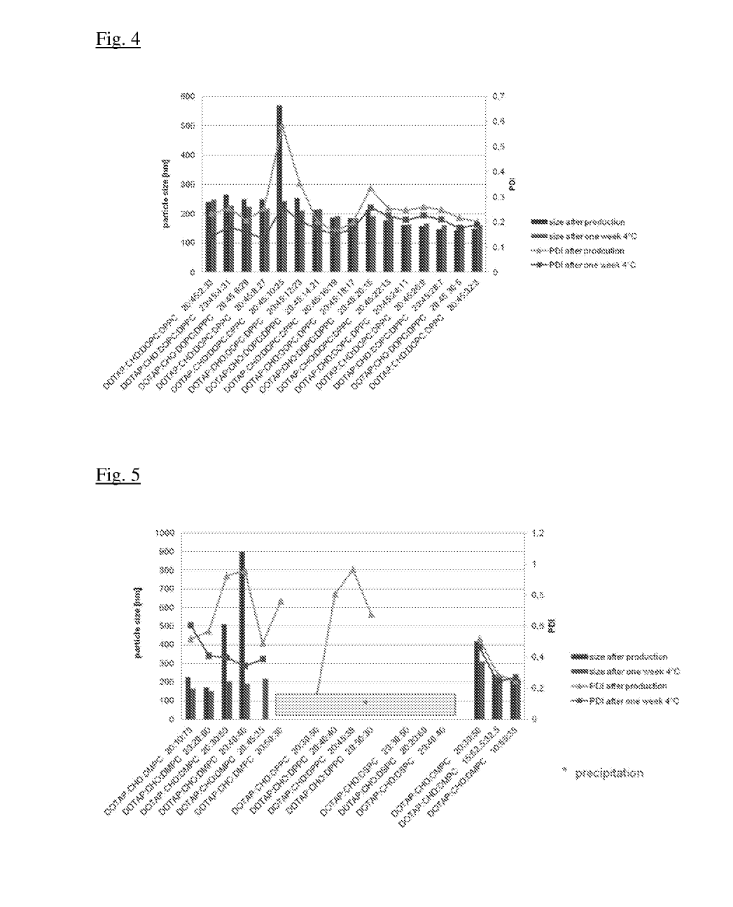

Apart from ternary mixtures also mixtures of DOTAP, cholesterol and one phospholipid more complex systems were investigated, especially focusing on simulating and replacing the fatty acid composition in the formulation component Lipoid EPC. As major PCs this mixture contains DSPC, DPPC, POPC and DOPC. Two limit complexity EPC was replaced by mixtures of two of these formulation components. FIGS. 2, 3 and 4 exemplify the influence of the helper lipid composition on formulation feasibility and stability of liposomes. Whereas DSPC shows limited suitability in terms of formulation characteristics mixtures of DOPC and DPPC or DMPC and DPPC demonstrate good formulation stability. DPPC and DOPC were titrated over the relevant concentration range and demonstrated only little variation in PSD when titrating DOPC from 2-32% (mol/mol) and DPPC from 3-32%, respectively.

### PEGylated Liposomes and Reduction of Salt Concentration

The concentration of monovalent ions proved to be critical with regard to dispersion stability in preliminary trials. While not being critical in liquid formulations, lyophilized or frozen formulations suffer from freezing point depressions at the salt content of 150 mM KCl. It was found that—in contrast to standard PCs—the presence of pegylated phospholipids in the liposome bilayer reduced the sensitivity of the formulation towards lowered salt content. FIG. 5 is displaying formulation trials at 100 mM KCl (standard 150 mM) and the dramatic effects on protein/formulation precipitation under this condition. In contrast, FIG. 6 is giving an overview of formulation options at a salt content of 50 mM KCl. It can be seen that introducing as low as 3% (mol/mol) DSPE-PEG2000 in the formulation allows the production of survivin loaded liposomes. At 1-2% DSPE-PEG2K the formulations show precipitation and pronounced increase in particle size distribution (PSD).

These results show (FIG. 6), as already mentioned earlier, that pegylation of phospholipids or liposomal particles contributes to the stability of the liposomal formulation with regard to the possible precipitation of the truncated survivin according to the invention. However, unfortunately pegylated liposomal particles containing survivin show a reduced anti-tumor efficacy and a decreased CD4⁺/CD8⁺ T cell activity as compared to non-pegylated particles of the same contents and composition.

### Freezing and Lyophilization of Liposomes

Three different formulations were tested for freezing at −70° C. and lyophilization:

| | |
|---|---|
| DOTAP/EPC/CHO | 20/35/45 (% ratio) |
| DOTAP/CHO/DOPC/DSPE-PEG2K | 20/45/32/3 (% ratio) |
| DOTAP/CHO/DOPC/DSPE-PEG2K | 20/45/30/5 (% ratio) |

Feasibility tests of different cryoprotectants (e.g., trehalose, or sucrose) showed superiority of sucrose in preserving liposome size distribution after lyophilization. Consequently, different sucrose concentrations were titrated in order to define the range of optimum size preservation. Concentrations were set to 0%, 2.5%, 5%, 7.5% and 10% sucrose (w/v).

About 10% sucrose was found to be optimal but interfered with in-vivo efficacy. Hence, frozen dispersions liposomes based on the lipid components DOTAP/CHO/EPC and 2-3% sucrose (w/w), preferably approximately 2.5% sucrose, elicit optimum physicochemical and in-vivo pharmaceutical activities.

Furthermore formulations containing PEG2K were found to be inferior to non-pegylated formations regarding immunological responses.

### In-Vivo Experiments

(i) Influence of DOTAP Concentration

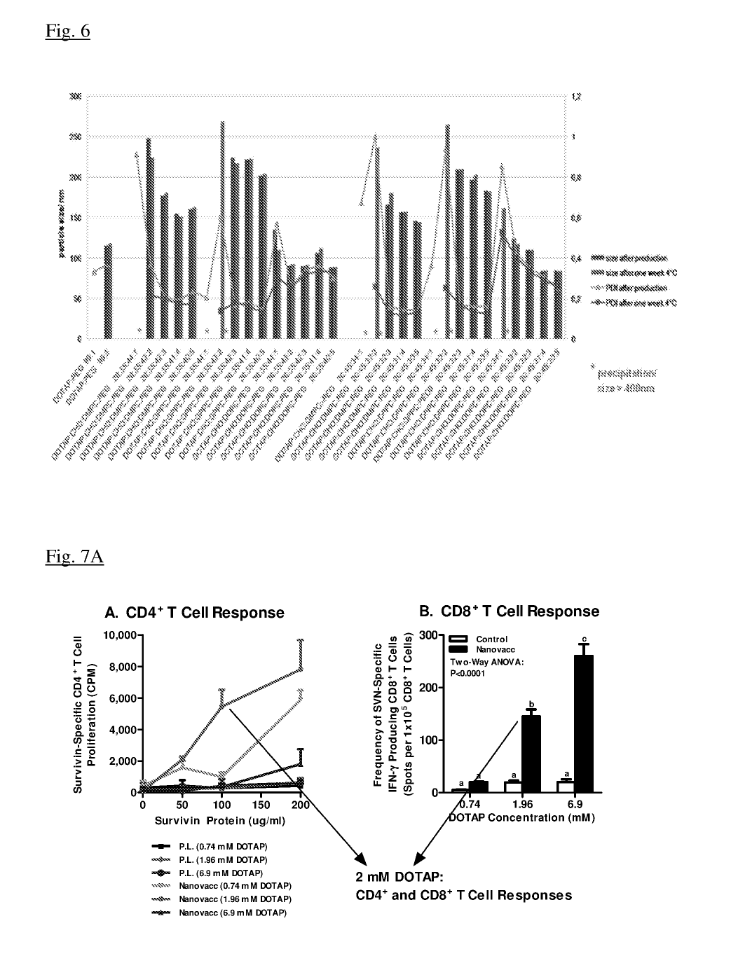

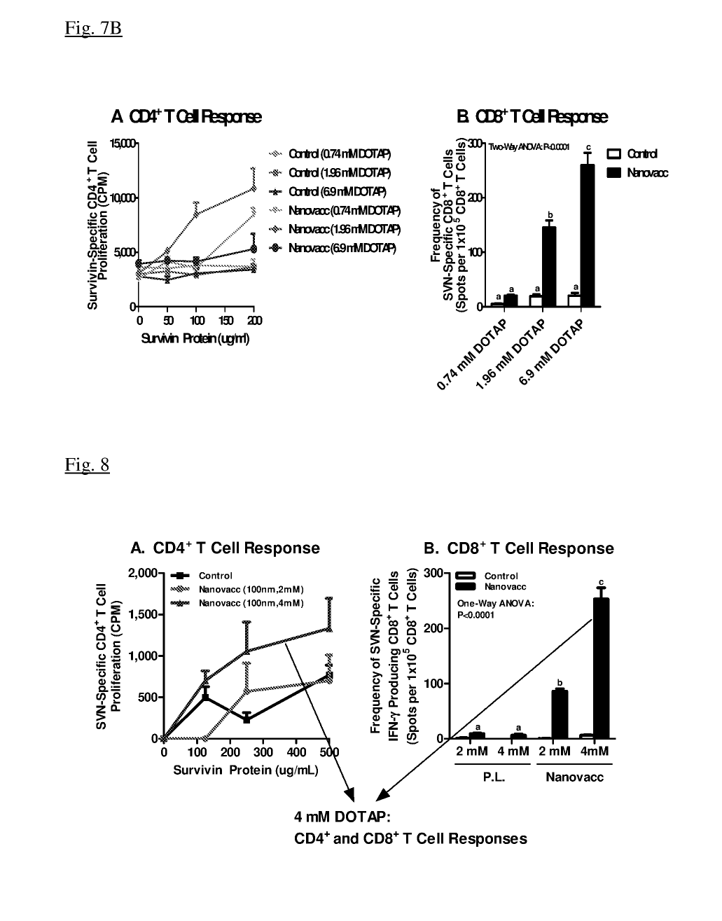

To investigate the influence of the DOTAP concentration per vaccination bolus the number of liposomes and thus, the concentration of DOTAP was varied in a range of 0.7 mM to 7 mM DOTAP. FIG. 7 displays the respective CD4⁺ and CD8⁺ T cell responses from mice vaccinated with either placebo liposomes (P.L.) or liposomes encapsulating the survivin protein. Whereas there a dose-dependent relationship was observed between the adjuvant content and CD8⁺ T cell responses (FIG. 7A), a loss of SVN-specific CD4⁺ T cell proliferation was noted at the highest DOTAP concentration. The results indicate an optimum of DOTAP concentration between 2 to 7 mM R-DOTAP with preferential skewing of the immune response toward SVN-specific CD8⁺ T cells at the highest concentration of DOTAP (7 mM). Moreover, the data in FIG. 7 demonstrate engagement of both CD4⁺ and CD8⁺ SVN-specific T cell responses at intermediate concentrations of DOTAP (e.g., 2 mM) in the vaccine formulation. To further elucidate the relevant concentration range a second study was set up comparing 2 mM and 4 mM R-DOTAP (FIG. 8). The results indicate a DOTAP concentration of 4 mM is superior to 2 mM at inducing SVN-specific CD4⁺ T cell proliferation (FIG. 8A) and IFN-γ producing CD8⁺ T cells (FIG. 8B).

(ii) Influence of Antigen Payload

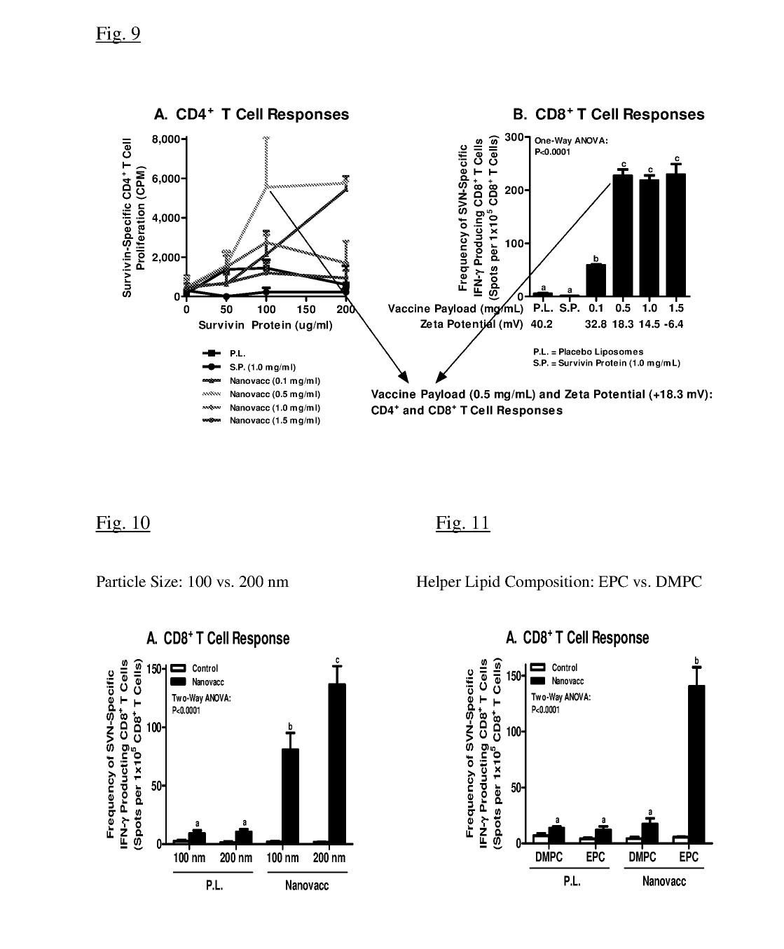

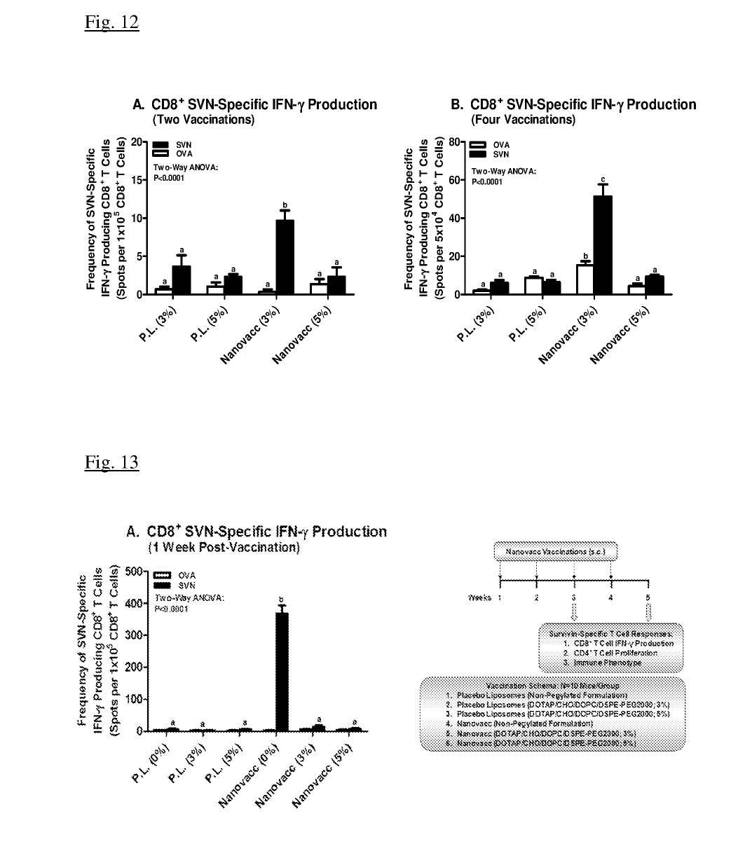

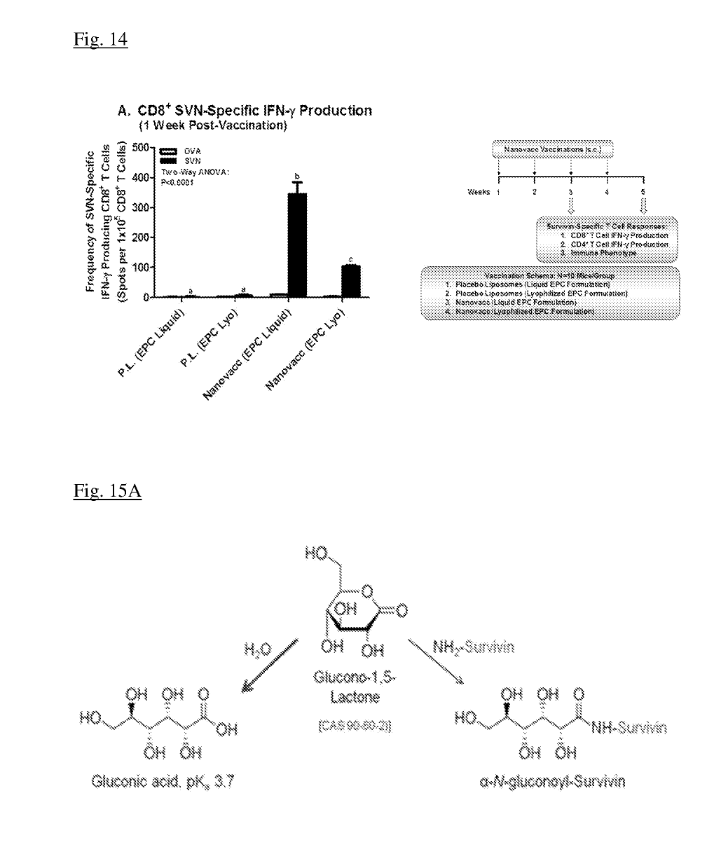

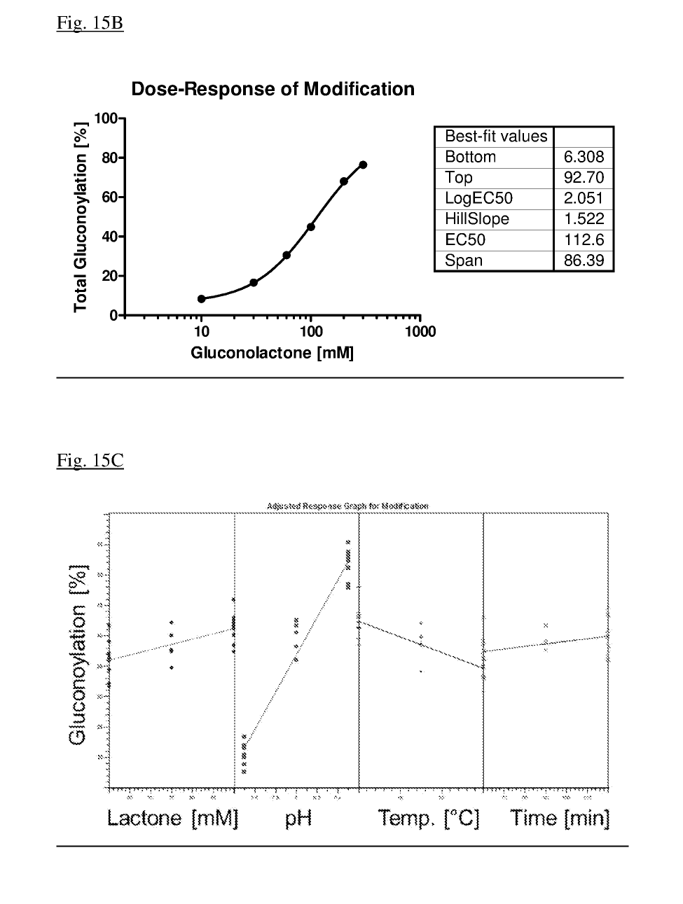

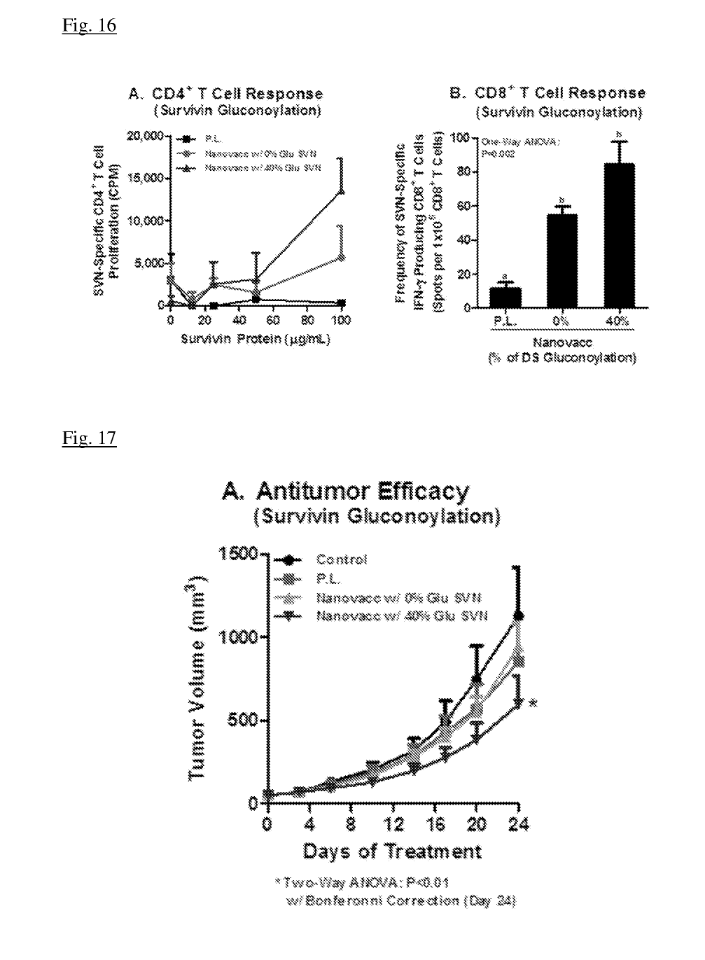

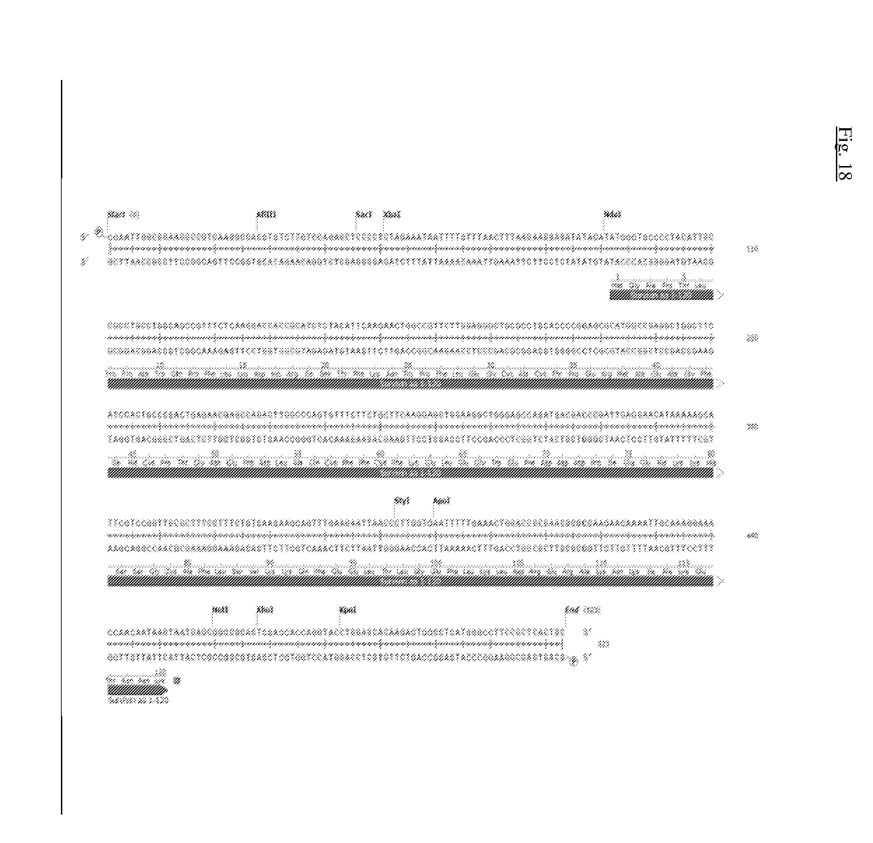

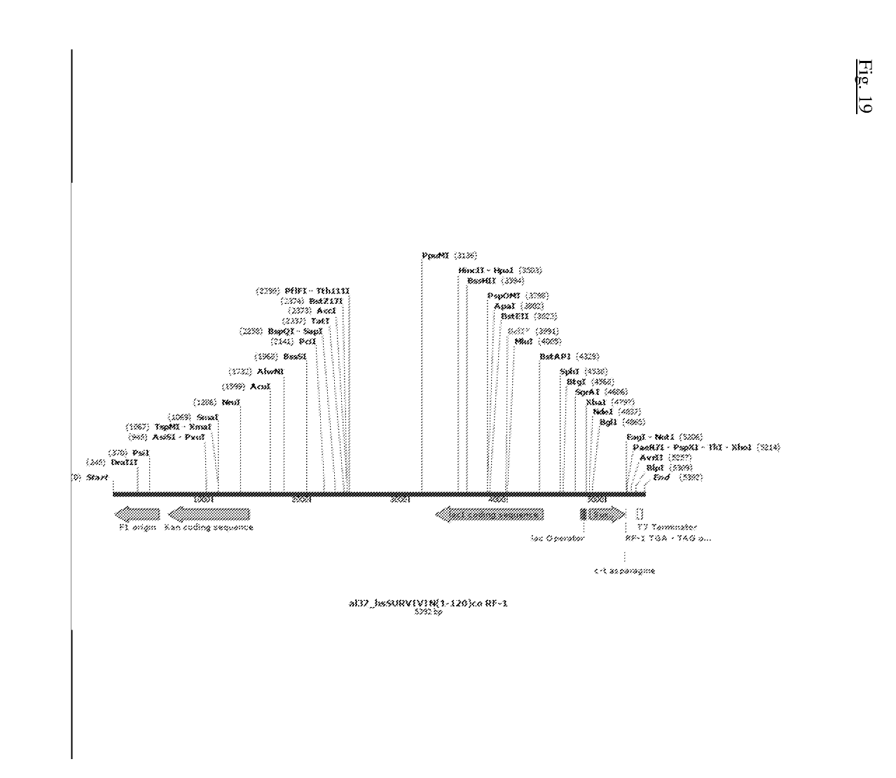

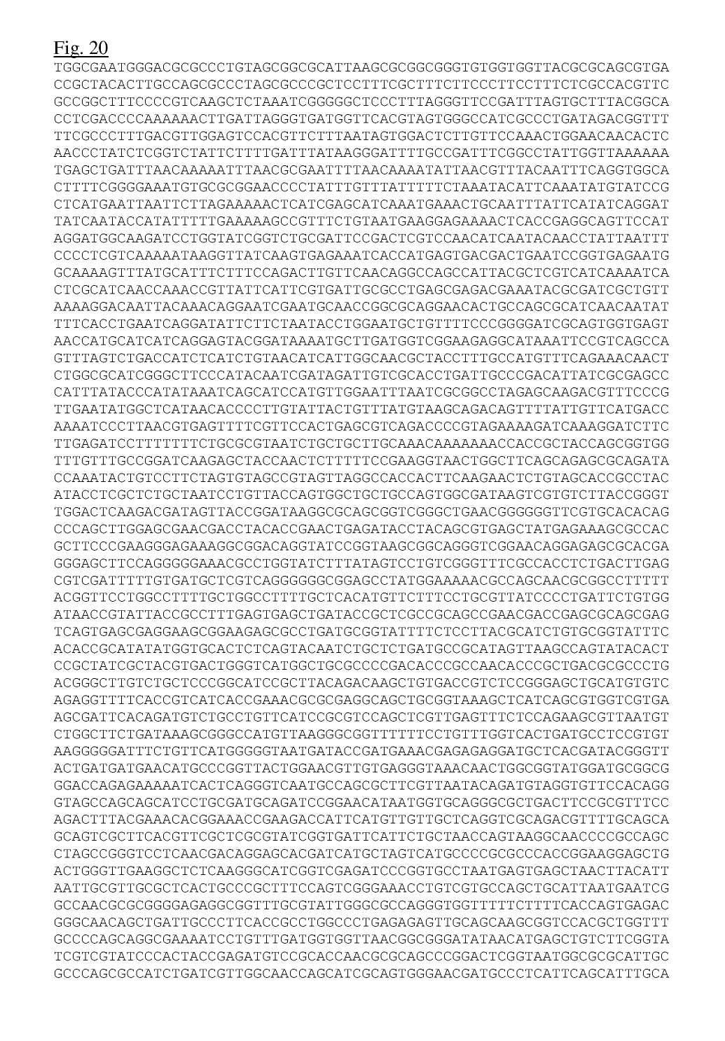

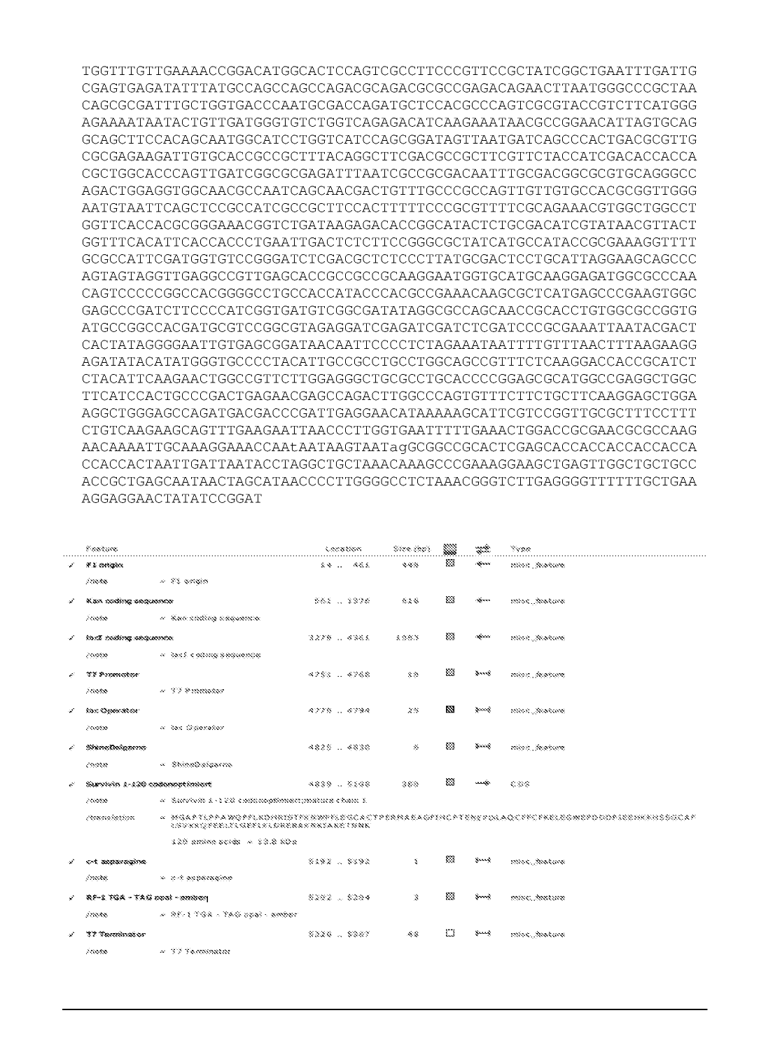

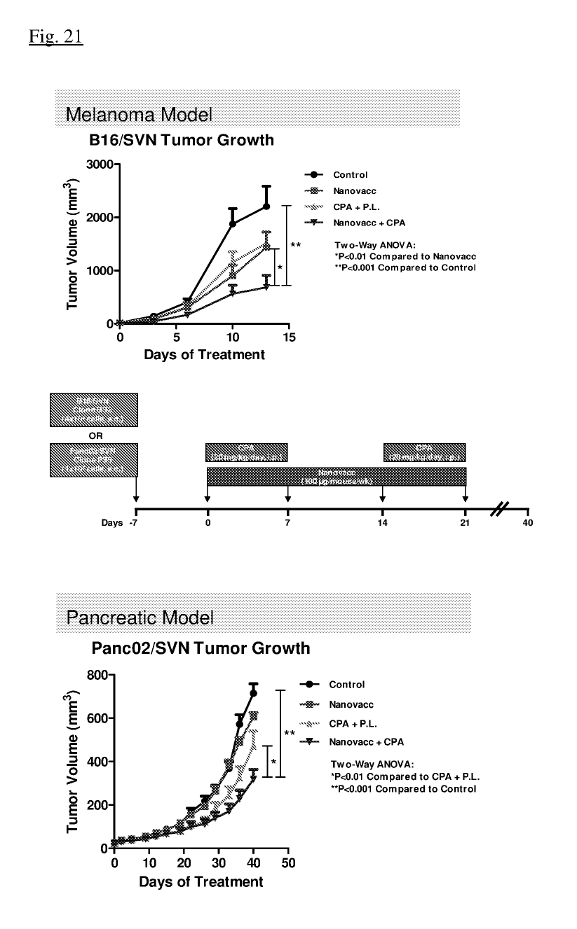

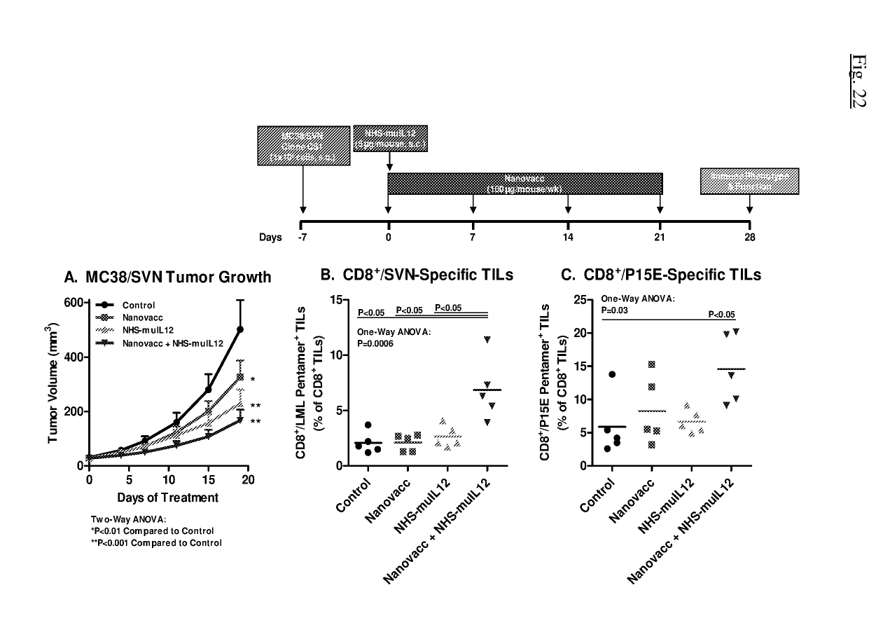

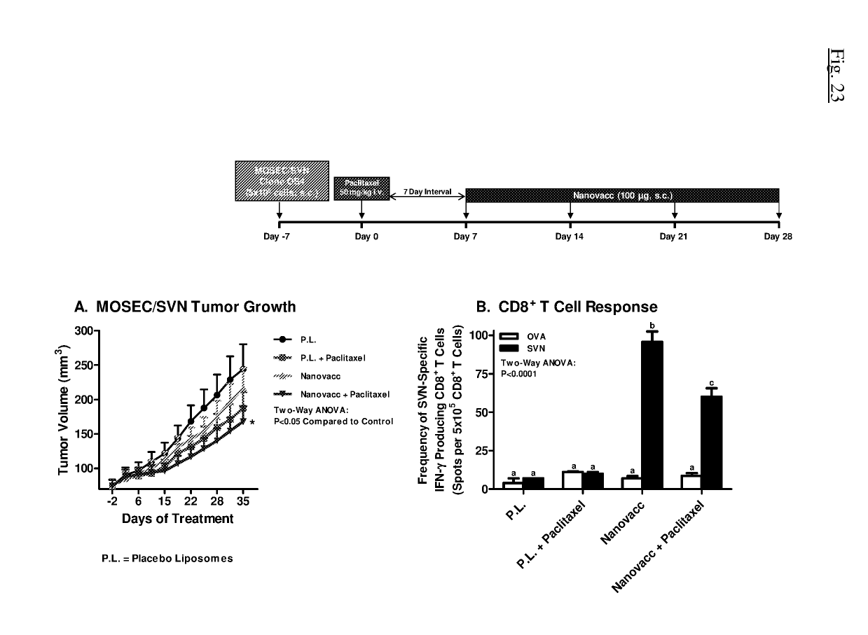

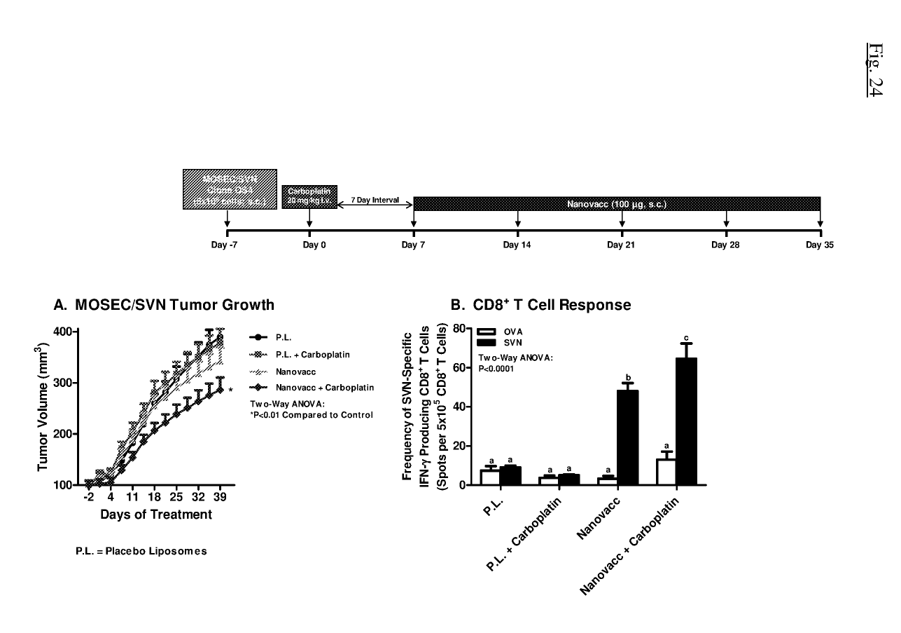

Liposomes according to the invention (e.g., 100 nm liposomes, 10 mM lipid, 20% DOTAP, 35% EPC, 45% cholesterol) were loaded with surviving to different degrees (0, 0.1 mg/mL, 0.5 mg/mL, 1.0 mg/mL, and 1.5 mg/mL) with deltaC (truncated) survivin (SEQ ID NO 3). CD4/CD8 responses suggest a payload optimum between 0.5-10.0 mg/mL (FIG. 9A) whereas a plateau in cytotoxic effect seems to be reached between 0.1-0.5 mg/mL antigen (FIG. 9B). Notably, neither placebo liposomes nor pure protein elicit any significant immunological effect.

(iii) Influence of Particle Size

Liposomes according to the invention (e.g., 100 nm versus 200 nm liposomes) were compared regarding the impact of different particle size in vaccine bioactivity. FIG. 10 displays the difference in CD8⁺ activation after two vaccinations. Liposomes having a particle size of 180-350 nm, preferably 200-250 nm, significantly augment immune responses within the test setting. The results suggest a particle size of around 200 nm being very suitable for vaccine applications.

(iv) Influence of Lipid Composition

Due to project history all early trials were performed with liposomes composed of 20% DOTAP/35% EPC/45% cholesterol (mol/mol). EPC is a rather crude mixture of different phosphatidylcholines, mainly consisting of PCs with C16, C18 and C18:1 fatty acids. Significant efforts were made in defining other formulation compositions enabling superior physicochemical stability of the liposomes and reduced complexity of the formulation. EPC could be efficiently replaced by DMPC as phospholipid component while it was possible to maintain all other formulation parameters, such as size, surface charge and antigen payload. The proposed formulation consisted of 20 mM 20% DOTAP/50% DMPC/30% cholesterol (mol/mol) and was subjected to immunologic animal studies. Data demonstrated a complete loss of immunologic activity when replacing EPC with synthetic helper lipids (FIG. 11).

In a second set of experiments the immunogenicity of pegylated liposomes was evaluated. Liposomes consisted of either 20% DOTAP/45% cholesterol/32% DOPC/3% DSPE-PEG2000 or 20% DOTAP/45% cholesterol/30% DOPC/5% DSPE-PEG2000, and they were tested against standard EPC-containing liposomes (FIG. 12). CD8 responses were recorded after 2 and 4 vaccinations. Data suggest that 1) 3% pegylation is superior in eliciting specific cytotoxic responses, and 2) immune responses are further augmented after 4 vaccinations. Similar to DMPC containing liposomes the frequency of CD8⁺ T cells was far lower than after vaccination with EPC containing liposomes. Liposomes are considerably smaller when introducing low amounts of DSPE-PEG2000. It was unclear if the loss of activity is predominantly mediated by the PEG shield on the particle surface or the reduced particle size that might lead to escape from immune system recognition. Therefore, another set of experiments with optimized particle size (˜200 nm) and DOTAP concentration (4 mM R-DOTAP) was conducted (see FIG. 13). Again, only mild immunoactivation can be detected in pegylated formulations whereas strong responses are observed for the cationic EPC-based formulation with 0% pegylation.

(v) Performance of Lyophilized Formulations

Due to the inherent instability of the surviving protein in aqueous environment alternatives for long term storage products need to be defined. A lyophilization protocol for preserving the physicochemical properties of the liposomes was developed. It included the addition of cryoprotectants to the formulation. A sucrose concentration of around 25-150 mg/mL, preferably 100-150 mg/mL, in the formulation buffer showed to efficiently preserve the relevant parameters (particle size, charge, drug payload) of the vaccine. FIG. 14 is depicting the vaccination protocol and the respective CD8⁺ readout of the reconstituted lyo formulation in comparison to a liquid formulation of equal lipid composition. It can be seen that cytotoxicity is compromised in the lyophilized formulation.

The results shown above clearly demonstrate that it is possible to formulate a 120 aa (deriving from SEQ ID NO: 1) truncated survivin variant in cationic liposomes. The formulation enables strong and reproducible adjuvant potency depending on the size and composition of the lipid particles, if DOTAP, preferably R-DOTAP is used. Formulation trials revealed that liposome stability and characteristics depend on the careful selection of formulation components. While pure R-DOTAP is not suitable to formulate the sensitive antigen, mixtures between DOTAP, cholesterol and a phosphatidylcholine component enabled the production of stable liposomes according to the target profile. Of special relevance was the introduction of pegylated lipids in the liposome matrix, as these significantly reduced undesired electrostatic reactions between survivin and R-DOTAP and enabled production and storage with reduced content of monovalent ions.

In-vivo results emphasized the importance of formulation parameters for the adjuvant performance of the vaccine. 3-5 mM, preferably 3.5-4.5 mM, more preferably 4 mM R-DOTAP was identified as the optimum adjuvant concentration at a particle size of approximately 200 nm (150-300 nm). Also the lipid composition proved to be of outmost importance for the vaccine. Introducing DMPC as formulation component inactivated the vaccine whereas strong and robust effects could be detected with a mixture of DOTAP, EPC and cholesterol. The addition of pegylated lipids and sucrose compromised the efficacy of the vaccine. Still, cytotoxic effects could be recorded even at 100 mg/mL sucrose. In-vivo performance is enhanced via reducing the concentration of sucrose to 50-25 mg/mL.

### Combination Therapy

In a further aspect, administration of immunotherapeutic agents is favorable to be combined concurrently or sequentially with the liposomal vaccination composition according to the invention. Suitable immunotherapeutic agents according to the invention are, for example, anti-cancer antibodies, such as anti-VEGF® antibodies, anti-EGFR antibodies like bevacizumab, cetuximab, panitumumab, erlotinib, gefitinib and afatinib, or anti-PDL1 antibodies, such as disclosed in WO 2013/079174. The antibodies may be fused preferably via its C-terminal heavy chains to cytokines, such as IL-2, TNFa, IFNb, and IL12.

In a preferred embodiment of the invention, the known fusion proteins NHS-IL12 and NHS-IL2 (Selectikine), preferably NHS-IL12, are combined with the liposomal preparations of the invention. NHS-IL12 is a fusion protein consisting of the heavy-chains of the known human antibody NHS76 binding to the necrotic core of tumors via nuclear cells (“TNT” antibodies, see WO 2000/001822), wherein said antibody is covalently fused at its C-terminal to the p40 and/or p30 subunits of (modified) IL-12, and is equipped with potential immunostimulating and antineoplastic activities. Upon administration, the antibody moiety of immunocytokine NHS-IL12 binds to DNA released from necrotic tumor cells located primarily at the core of necrotic solid tumors, thereby delivering the IL-12 moiety. In turn, the IL-12 moiety of this agent stimulates the host immune system to mount an immune response against tumor cells, thereby inhibiting tumor growth. IL-12 is a proinflammatory cytokine with numerous immunoregulatory functions and may augment host immune responses to tumor cells. By targeting tumor cells, NHS-IL-12 may reduce the toxicity associated with systemic administration of recombinant human IL-12.

Preferred combination therapies based on truncated survivin according to the invention for treatment of a diversity of cancers, preferably ovarian cancer include (FIG. 21-24):
Combination with low dose cyclophosphamide (CPA): Low dose CPA was found to enhance immune responses and this is known to be mediated through the depletion of CD4_25-Treg cells. The activities of CPA were also observed in several cancer vaccine clinical trials. In DPX-Survivac phase I study, it was also shown metronomic low dose CPA could directly enhance the immunogenicity of DPX-Survivac.
Combination with anti PD-L1: The common rationale for the vaccine is the activation of APCs and the stimulation of an antigen-specific CTL mediated immune response. However, in the setting of chronic tumor antigen stimulation, CD8 T cells undergo exhaustion, causing them to become dysfunctional. While multiple mechanisms contribute to the process of exhaustion, PD-L1 is the most well characterized inhibitory molecule up-regulated and is associated with disease progression and immune dysfunction. The blockage of PD-L1 has showed the effectiveness for increasing tumor clearance in clinical trials. Therefore, the assumption is that the combination of the present invention with anti PD-L1 will improve CTL responses. Furthermore, anti-PD-L1 has been studied in ovarian cancer patients who failed at least one line of chemotherapy. 18% of ovarian cancer patients (n=17) were able to achieve stable disease at least 6 months.
Combination with NHS-ID 2: NHS-IL12 is a targeted delivery of IL-12 which is supposed to make this cytokine a safer, more effective cancer therapy. IL-12 is involved in the differentiation of naive T cells into Th1 cells which can stimulate the growth and function of T cells. It stimulates the production of IFN-γ and TNF-α from T and NK cells, and reduces IL-4 mediated suppression of IFN-γ. IL-12 also has anti-angiogenic activity, which means it can block the formation of new blood vessels. Therefore, combination of the truncated survivin according to the invention with above specified drugs or other anti-cancer drugs is supposed to enhance overall anti tumor activities.

The vaccine compositions of the invention can also additionally contain further compounds, which are known to be immune-stimulating, such as CpGs or cylcophosphamide. The CpG nucleic acid preferably contains at least one or more (mitogenic) cytosine/guanine dinucleotide sequence(s) (CpG motif(s)). In certain embodiments, the liposomal preparations or vaccine compositions according to the invention function in concert with an effector molecule that contributes to the immune response. For example, such an effector molecule can be a cytokine moiety including, but not limited to, IL-2, IL-7, IL-12, IL-18, IL-21, IL-23, GM-CSF, or any other cytokine, particularly one capable of activating a Th1 immune response. Such an effector molecule can also be an inhibitor of a cytokine that represses the immune system, for example, a STAT3 inhibitor.

### Therapeutic Use

In one aspect, the invention relates to the use of the liposomal preparation as described herein or an immunostimulatory vaccine as described herein for the manufacture of a medicament for the prophylaxis or treatment of cancer or cancer-related diseases, wherein the cancer or cancer-related disease is based on the presence of survivin-specific tumor cells. In a preferred aspect, said liposomal preparation is used in combination with an anti-cancer agent selected from the group consisting of cyclophosphamide, carboplatin, paclitaxel and NHS-IL12.

In one aspect, the invention relates to a method of preventing or treating cancer or cancer-related diseases in an individual in need thereof, wherein the cancer or cancer-related disease is based on the presence of survivin-specific tumor cells in said individual by administering to said individual a therapeutically effective dose of an immunostimulatory vaccine as described herein. In a preferred aspect, the invention relates to said method of treatment by additionally administering an anti-tumor agent selected from the group consisting of cyclophosphamide, carboplatin, paclitaxel and NHS-IL12.

Due to the fact that survivin appears to be expressed in most cancer forms, it is very likely that the vaccine compositions or liposomal preparations of the invention can be provided to control any type of cancer disease in which survivin is expressed.

Thus, as examples, the composition and preparation of the invention is immunologically active against a hematopoietic malignancy including chronic lymphatic leukemia and chronic myeloid leukemia, melanoma, breast cancer, cervix cancer, ovary cancer, lung cancer, colon cancer, pancreas cancer and prostate cancer. The therapeutic use comprises administering to a patient suffering from the disease an effective amount of the pharmaceutical composition according to the invention.

According to the present invention, the inventive vaccine composition or liposomal preparation may additionally contain a pharmaceutically acceptable carrier and/or further auxiliary substances and additives and/or adjuvants.

The inventive preparation typically comprises a safe and effective amount of the truncated survivin polypeptide as described above. As used herein, “safe and effective amount” means an amount of truncated survivin according to the invention, which is sufficient to significantly induce a positive modification of cancer, for example, lung or ovarian cancer. At the same time, however, a “safe and effective amount” is small enough to avoid serious side-effects, and that is to say to permit a sensible relationship between advantage and risk. The determination of these limits typically lies within the scope of sensible medical judgment. In relation to the drug substance of the invention, the expression “safe and effective amount” preferably means an amount of truncated survivin according to the invention that is suitable for stimulating the adaptive immune system in such a manner that no excessive or damaging immune reactions are achieved but, preferably, also no such immune reactions below a measurable level. A “safe and effective amount” will furthermore vary in connection with the particular condition to be treated and also with the age and physical condition of the patient to be treated, the severity of the condition, the duration of the treatment, the nature of the accompanying therapy, of the particular pharmaceutically acceptable carrier used, and similar factors, within the knowledge and experience of the accompanying doctor. The liposomal or vaccine preparation according to the invention can be used according to the invention for human and also for veterinary medical purposes, as a pharmaceutical composition for vaccination.

The expression “pharmaceutically acceptable carrier” as used herein preferably includes the liquid or non-liquid basis of the inventive liposomal or vaccine preparation. If the liposomal preparation or the vaccine composition according to the invention is provided in liquid form, the carrier will typically be pyrogen-free water; isotonic saline or buffered (aqueous) solutions, e.g., phosphate-, or citrate-buffered solutions.

The amount of the immunogenic vaccine composition of the invention may vary, depending on the particular application. However, a single dose of the vaccine is preferably anywhere from about 10 μg to about 5000 μg, more preferably from about 50 μg to about 2500 μg, such as about 100 μg to about 1000 μg. In a preferred embodiment of the invention one single dose of the liposomal formulation should contain, according to the invention, 500-1.200 μg of said lipopeptide, more preferably 700-900 μg. In a preferred embodiment of the invention, the vaccination by means of the liposomal preparation of the invention is accompanied by the administration of cyclophosphamide between 100-400 mg/m², preferably 250 mg/m², by which the immune system of the patient can be activated or enhanced. Usually, a single dose before start of the vaccination, as a rule 1 to 5 days, preferably 2-5 days, should be sufficient to be effective, however other different regimens are applicable.

Modes of administration include intradermal, subcutaneous and intravenous administration, implantation in the form of a time release formulation, etc. Any and all forms of administration known to the art are encompassed herein. Also any and all conventional dosage forms that are known in the art to be appropriate for formulating injectable immunogenic peptide composition are encompassed, such as lyophilized forms and solutions, suspensions or emulsion forms containing, if required, conventional pharmaceutically acceptable carriers, diluents, preservatives, adjuvants, buffer components, etc.

In a further aspect of the invention, the vaccine compositions and liposomal preparations according to the invention can be combined with other pharmaceutically effective agents and drugs discussed in more detail in the following.

### Chemo-/Radiotherapy

The vaccination approach according to the invention also comprises “chemo-radiotherapy”. Chemo-radiotherapy according to the invention includes “chemotherapy”. Chemo-radiotherapy also includes “radiotherapy” carried out by radiation according to standard methods or by administration of radio-labeled compounds. According to the invention radiation is preferred.

The term “cytotoxic agent” as used herein refers to a substance that inhibits or prevents the function of cells by causing destruction of cells. The term is intended to include radioactive isotopes, chemotherapeutic agents, immunotherapeutic agents, and toxins, such as enzymatically active toxins of bacterial, fungal, plant or animal origin, or fragments thereof. The term may include also members of the cytokine family, preferably IFNγ as well as anti-neoplastic agents having also cytotoxic activity.

The term “anti-cancer agent” or “anti-tumor agent” describes all agents which are effective in cancer/tumor therapy. The term includes, cytotoxic agents, chemotherapeutic agents, and immunotherapeutic agents.

Chemo-radiotherapy according to the invention usually starts with chemotherapy followed by radiotherapy. However, starting therapy with radiotherapy is also applicable. Chemotherapy is carried out by administration of at least one “chemotherapeutic agent”, preferably a platinum-based drug, such as cisplatin or carboplatin. According to the invention, chemotherapeutic agents are administered daily, weekly or every 2 to 5 weeks, which is dependent on the dose duration and number of administrations.

Chemotherapy according to the invention comprises administration of chemotherapeutic agents which are according to the understanding of this invention a member of the class of cytotoxic agents, and include chemical agents that exert anti-neoplastic effects, i.e., prevent the development, maturation, or spread of neoplastic cells, directly on the tumor cell, and not indirectly through mechanisms, such as biological response modification.

Preferred chemotherapeutic agents according to the invention, which are administered in the chemo-radiotherapy settings of the invention, are platinum-based agents, such as cisplatin or carboplatin. However, other chemotherapeutic agents as specified below may be also used.

In addition, further chemotherapeutic agents or other anti-cancer agents can be administered to improve efficacy of the claimed therapy. There are large numbers of anti-neoplastic agents available in commercial use, in clinical evaluation and in pre-clinical development, which could be included in the present invention for treatment of tumors/neoplasia by combination therapy. It should be pointed out that the chemotherapeutic agents can be administered optionally together with above-said liposomal preparations. Examples of chemotherapeutic or agents include alkylating agents, for example, nitrogen mustards, ethyleneimine compounds, alkyl sulphonates and other compounds with an alkylating action, such as nitrosoureas, cisplatin and dacarbazine; antimetabolites, for example, folic acid, purine or pyrimidine antagonists; mitotic inhibitors, for example, vinca alkaloids and derivatives of podophyllotoxin; cytotoxic antibiotics and camptothecin derivatives. Preferred chemotherapeutic agents or chemotherapy include amifostine (ethyol), cabazitaxel, cisplatin, dacarbazine (DTIC), dactinomycin, docetaxel, mechlorethamine, streptozocin, cyclophosphamide, carrnustine (BCNU), lomustine (CCNU), doxorubicin (adriamycin), doxorubicin lipo (doxil), gemcitabine (gemzar), daunorubicin, daunorubicin lipo (daunoxome), procarbazine, ketokonazole, mitomycin, cytarabine, etoposide, methotrexate, 5-fluorouracil (5-FU), vinblastine, vincristine, bleomycin, paclitaxel (taxol), docetaxel (taxotere), aldesleukin, asparaginase, busulfan, carboplatin, cladribine, camptothecin, CPT-11, 10-hydroxy-7-ethyl-camptothecin (SN38), dacarbazine, floxuridine, fludarabine, hydroxyurea, ifosfamide, idarubicin, mesna, interferon alpha, interferon beta, irinotecan, mitoxantrone, topotecan, leuprolide, megestrol, melphalan, mercaptopurine, plicamycin, mitotane, pegaspargase, pentostatin, pipobroman, plicamycin, streptozocin, tamoxifen, teniposide, testolactone, thioguanine, thiotepa, uracil mustard, vinorelbine, chlorambucil, and combinations thereof.

In a preferred embodiment of the invention a liposomal formulation is provided, wherein the chemotherapeutic agent is selected from the group consisting of cisplatin or carboplatin, and the non-platinum based chemotherapeutic agent is selected from the group consisting of vinorelbine, etoposide, paclitaxel, docetaxel, vindesine, gemcitabine, ifosfamide and pemetrexed, preferably paclitaxel.

Chemotherapy is applied according to the invention by usually at least two cycles, preferably 2-8 cycles, more preferably 2-5 cycles. One cycle is between 21 and 35 days, preferably between 21-28 days. The dose regimen of the chemotherapeutic agent is dependent on various possible patient- and drug-related conditions and properties. Usually, cisplatin is applied in doses varying from 50-120 mg/m² and per cycle. Carboplatin or paclitaxel may be applied according to the invention in doses of 100-1500 mg per single dose and per cycle.

Radiotherapy is carried out according to the invention by standard radiation, wherein a total of 40-120 Gy are applied, preferably at least 50 Gy, more preferably between 50 and 75 Gy. The radiation therapy is usually fractionated, wherein 1.5-3.5 Gy are applied per day for at least four days, preferably 5-7 days in sequence. The total radiation dose is to be applied according to the invention within 21-35 days, preferably within 28 days. If necessary or favorable, boost doses of 3.5-15 Gy, preferably 5-10 Gee can be applied at the beginning of radiation or in an intermediate interval.

The following examples describe the invention in more detail without limiting the scope of the invention.

### EXAMPLES

### Example 1

### Materials

The phospholipid species applied in this invention are listed in Table 1. DOTAP was obtained by Merck Millipore and cholesterol was purchased from Sigma Aldrich. All other lipids were obtained from Lipoid AG, Germany. dC-Survivin was manufactured as described below and consisted of the amino acid sequence of human survivin shortened at the C-terminus to achieve more preferable stability characteristics and better manufacturability. It contains the amino acids 2-120 of human survivin (SEQ ID NO: 3) and had the following characteristics: molecular weight 13.8 kDa, isoelectric point 6.0 and melting point 48° C.

### Example 2

### Engineering of Source Strain E. coli BL21(DE3) and Preparation of E. coli T7E2 Strain as Expression Strain for Truncated Survivins of the Invention

The source strain E. coli BL21(DE3) (Novagen) was engineered as follows:
Incoming analysis of E. coli BL21(DE3)
Altering the E. coli BL21(DE3) genomic rpsL gene to achieve a Streptomycin resistant phenotype
Replacement of the dispensable DE3 prophage region by a loxP-cmR-loxP cassette in the E. coli BL21(DE3) STR genomic background
Replacement of the Rac prophage by a synthetic pgl gene in the E. coli BL21(DE3) STR L′1DE3:loxP-cmR-loxP genetic background
Removal of the loxP-flanked chloramphenicol marker

The genome sequence of E. coli BL21(DE3) [gb#CPOO 1509 0.3] and Artemis software (Rutherford et al., Bioinformatics 16(10): 944-5, 2000) were used for in-silico strain engineering. E. coli BL21(DE3) was aerobically propagated on LB Vegitone broth and agar or maltose M9-Minimalplate (0.4%) supplemented with L-leucine (0.04 mg/mL). As necessary, ampicillin (Ap), chloramphenicol (Cm), kanamycin (Km), streptomycin (Str), and tetracycline (Tc) were added to final concentrations of 50 flg/mL, 15 flg/mL, 15 flg/mL, 50 flg/mL, and 3 flg/mL, respectively.

In order to ensure that the source strain E. coli BL21(DE3) (purchased from Novagen) does not gluconoylate, the strain genome DNA was bioengineered. The BL21(DE3) genome contains a number of pro-phages. There is a latent risk that under certain physiological conditions some of these may become active to generate infectious phage particles. The presence of the Lambda DE3 phage goes back to the introduction of the T7 polymerase (T7-RNAP) into the bacterial genome. To further reduce the chance of mobilization, 38.3 kilobases of the DE3 pro-phage sequence upstream of the T7-RNAP gene have been deleted in the expression strain. Further to this, the Rac pro-phage locus was deleted in course of introducing the phospho-gluconolactonase gene copy. The resulting strain according to the invention is E. coli T7E2 (#1) and is characterized by the following genetic traits: (i) Streptomycin-resistance due to a point mutation within genomic rpsL resulting in a Lys43Arg exchange (prerequisite for subsequent rpsL-based counter selection), (ii) markerless removal of 89.1% of the DE3-propage without impairing the T7 RNA-polymerase gene and function and (iii) replacement of the Rac-prophage by a synthetic pgl-gene derived from E. coli MG 1655 genome sequence.

### Example 3

### Transformation of E. coli T7E with Expression Plasmid pAL37

Source plasmid was pET42 (Novagen) which was finally designed via pAL28 to final expression plasmid pAL37. The plasmid DNA of pLA37 is fully depicted in FIG. 20 and represented by SEQ ID NO: 4. The restriction sites for the respective restriction enzymes are depicted in FIG. 19. Plasmid AL37 contains the DNA coding for the truncated survivin sequence SEQ ID NO: 2 (1-120 aa of SEQ ID NO: 1) from which after expression and release into the medium the first methionine residue was cleaved off, thus representing truncated survivin SEQ ID NO: 3 (2-120 aa of SEQ ID NO: 1).

For the transformation, E. coli T7E2#1 cells were thawed on ice, 1 μl of plasmid al37 was added and incubated for 30 minutes on ice, incubated for 30 seconds at 42° C. and again incubated 2 minutes on ice. Then, 250 μl antibiotic-free LB vegitone broth were added and incubated for 60 minutes at 37° C., shaking at 450 rpm. The culture was plated on a LB vegitone agar plate supplemented with kanamycin and streptomycin and incubated at 37° C. over night.

Cells from this plate were used to generate an agar plate with single colonies by threefold streaking. After two days of incubation at 28° C. a s mall single colony was used to inoculate 200 mL chemically defined medium in a 1 L shake flask with baffles at 28° C. and 160 rpm over night for the first culture. The second pre-culture was inoculated with 1.44 mL of the first culture for an optical density of OD 578 nm=0.001. The culture was incubated at 28° C., 160 rpm, for approximately 20 hours. 17% glycerol and 2 mM betaine was added and the cells suspension distributed in 10×1 mL cryovials, the lids closed and the vials shockfrosted in liquid nitrogen and stored above liquid nitrogen.

For the generation of the research working cell banks, in each case, one vial of the first cell stock was thawed. 175 μl of the stock was used to inoculate 200 mL chemically defined medium in a 1 L shake flask with baffles at 28° C., 160 rpm and incubated over night. 17% glycerol and 2 mM betaine was added, and the culture distributed in 50×1 mL cryovials for the first and 100×1 mL vials for the second RWCB. The lids were closed and the vials were placed in the cell freezing device Planer Kryo 10 and frozen. After the freezing process was finished the vials were carefully placed in a cryotank. The resulting material was analyzed for identity, purity, viability, Gram staining and contaminating phages. All acceptance criterions were met.

### Example 4

### Protocol of Gluconoylation of Truncated Survivin According to the Invention

Gel filtration of the truncated survivin obtained from Example 3 into buffer
125 mg Survivin (5 mg/mL) stirred in water bath at 25° C.
Addition of solid glucono-1,5-lactone, 200 mM final
Time course 0-20 h, main reaction withdrawn after 45 min
Gel filtration
Concentration
Analysis by cIEF, ESI-MS, SE+RP-HPLC
Yield of approx. 100 mg (80%) with total gluconoylated fraction of ˜65%
Reaction seizes after approx. 60 min (pH indicates complete hydrolysis of GL)
No difference between mono and double modification kinetics at this resolution

### Example 5

### Liposome Production Method

Buffer or protein solution of 220 μl was added into each well of the 96-well plate. Lipid was first dissolved in ethanol and then injected into the buffer-containing wells at 33 μl/well. Each formulation was repeated three times. Each well was mixed by pipetting. Lipid injection was done by the Eppendorf Multipette Stream at dispense mode and speed level 5, which correlates to approximately 0.5 mL/min for aqueous solutions. The final mixing step was done by Biohit Proline 12-Channel pipette, at P mode and speed level 1, which is approximately 30 mL/min. The volume per pipetting for mixing was 100 μl and each well was pipetted once. Unless otherwise noted, the final lipid concentration was 1 mM and the survivin concentration was 0.1 mg/mL. Each formulation was repeated three times.

### Example 6

### Continuous Ethanol Injection Liposome Production Method

Buffer or protein solution was pumped into the sample vessel at a flow rate of 80 mL/min, using a silicone tube of 2 mm inner diameter. Lipid was first dissolved in ethanol and then injected into the buffer flow at a flow rate of 12 mL/min. The final liposome solution was collected in the sample vessel. Unless otherwise stated, final formulations exhibited a lipid concentration of 10 mM and a total protein content of 1 mg/mL. Each formulation was repeated three times.

### Example 7

### Particle Size and Zeta Potential Measurement

The liposomes were sized by dynamic light scattering, using the Malvern Zetasizer Nano-ZS. Measurements were done under 25° C. at fixed backscat tering angle of 173°. The zeta potential of liposomes was measured by the same Malvern Zetasizer Nano-ZS instrument. The liposome solution was diluted by 1:100 in water. Measurement was done at 25° C. using the Smoluchowski model.

### Example 8

### Formulation of the Drug Substance Generating the Drug Product According to the Invention

Liposomes dispersed in buffer medium consisting of the following components:
Lipid composition: 20% R-DOTAP, 35% high purity EggPC (EPC), 45% cholesterol (all % mol/mol)
Total lipid composition in dispersion: 20 mM
Dispersion medium: 20 mM K-Phosphate pH7, 150 mM KCl, 10 mM monothioglycerol (MTG)
Content dC-Survivin: 0.8-1.0 mg/mL total content, drug load 0.6 mg/mL

An ethanolic solution of phospholipids containing 77 mM lipids was prepared by weighing the 107.6 mg DOTAP, 207.1 mg EPC and 134.0 mg cholesterol in a suitable glass beaker and adding ad 10 mL with ethanol (>99% purity). The API was diluted in a buffer containing 20 mM K₂HPO₄, 150 mM KCl, 10 mM MTG.

Liposomes were prepared via a controlled mixing of the ethanolic and buffer solution in a t-shaped mixing device. The flow rates were 12 mL/min for the ethanol phase and 80 mL/min for the buffer phase. The resulting liposome dispersion was purified via dialysis in a Slide-A-Lyzer dialysis cassette (MWCO 10 kDa, PES) for 24 h with 5× buffer exchange. In a last step, the liposomes were concentrated 2× via diafiltration. The resulting liposomes were measured via DLS and show an average particle size of app. 120 nm and a PDI of <0.15. For further processing the liposomes were either frozen or freeze-dried in the presence of 2.5% (w/v) sucrose. Resulting particles showed an average particle size of 200-300 nm with a PDI>0.15. Drug load analysis revealed that 0.6 mg/mL of antigen were bound to the liposome particles.

In order to enable lyophilization of the samples, the buffer composition was changed towards 20 mM K₂HPO₄. It also required the adaptation of the cation towards potassium in the monovalent salt. Titration of KCl was performed from 0-150 mM in a buffer consisting of 20 mM K₂HPO₄, 10 mM MTG, pH 7.0. A minimum of 30 mM KCl was required to preserve the protein in the DS buffer. In order to ensure processability in presence of liposomes, the concentration was increased to 150 mM KCl.

1. A C-terminally truncated survivin consisting of the first 118-133 N-terminal amino acids of full-length human survivin represented by SEQ ID NO: 1, or a sequence having a homology to said truncated survivin of >90%, wherein the truncated survivin is gluconylated at one or more positions having free amino residues.

2. The truncated survivin according to claim 1, wherein at least one of the lysine residues is gluconoylated.

3. The truncated survivin according to claim 1 or 2, which consists of SEQ ID NO: 2 or SEQ ID NO: 3.

4. (canceled)

5. (canceled)

6. (canceled)

7. The truncated survivin according to claim 3, wherein the glycine residue at position 1 of SEQ ID NO: 3 and the lysine residues at position 22 and/or position 102 of SEQ ID NO: 3 are gluconoylated.

8. (canceled)

9. (canceled)

10. A survivin composition comprising the truncated survivin according to any of claims 1, 2, 3 or 7 and the same truncated but non-gluconoylated survivin, wherein in said composition 10-80% of the truncated survivin molecules are gluconoylated.

11. (canceled)

12. A liposomal preparation comprising the truncated survivin according to any of claims 1, 2, 3 or 7, or the survivin composition according to claim 10, and at least one adjuvant, wherein the at least one adjuvant is a chiral cationic phospholipid acting as immunomodulator.

13. The liposomal preparation according to claim 12, wherein the chiral cationic phospholipid is 1,2-dioleoyl-3-trimethylammonium propane (DOTAP), preferably R-DOTAP and its content in the lipid preparation varies between 15 and 30% (mol/mol).

14. (canceled)

15. (canceled)

16. The liposomal preparation according to claim 12 or 13, further comprising cholesterol and at least one phospholipid selected from the group consisting of phosphatidylcholine (PC), phosphoethanolamine (PE), and phosphoglycerol (PG).

17. (canceled)

18. (canceled)

19. (canceled)

20. The liposomal preparation according to any of claim 12, 13 or 16, further comprising cholesterol of a content of 25-55% (mol/mol) and/or the PC of a content of 30-80% (mol/mol).

21. (canceled)

22. The liposomal preparation according to any of claim 12, 13, 16 or 20, wherein the liposome particles of the preparation have a particle surface charge of ≧17 mV dependent on an antigen payload, which is 0.5-1.5 mg/mL, and/or a particle size between 40 and 500 nm.

23. (canceled)

24. (canceled)

25. The liposomal preparation according to any of claim 12, 13, 16, 20 or 22, comprising:
(i) the survivin composition according to claim 10,
(ii) the lipid components R-DOTAP, cholesterol and egg phosphatidylcholine EPC,
(iii) a R-DOTAP concentration in said preparation of 3-5 mM,
(iv) liposomal particles, wherein the particle size of at least 70% of the particles is 50-250 nm, and
(v) an antigen payload of 0.5 mg/mL.

26. The liposomal preparation according to claim 25, wherein
(i) the truncated survivin is represented by SEQ ID NO: 3 and non-glycosylated, wherein 40% (±5%) of said truncated survivin is gluconoylated, and
(ii) the contents of the lipid composition are:
10-30% R-DOTAP,
25-55% cholesterol, and
30-60% EPC (mol/mol).

27. An immunostimulatory vaccine comprising the liposomal preparation according to any of claim 12, 13, 16, 20, 22, 25 or 26, further comprising pharmaceutically acceptable carriers, excipients, additives or diluents.

28. The immunostimulatory vaccine according to claim 27 for use in the prophylaxis or treatment of a human individual by vaccination, wherein the individual suffers from cancer or a cancer-related disease wherein the cancer or cancer-related disease is based on the presence of survivin-specific tumor cells, and wherein the vaccination triggers survivin-specific immune responses in said individual by enhancing CD4⁺ and/or CD8⁺ T-cell responses.

29. (canceled)

30. (canceled)

31. (canceled)

32. The immunostimulatory vaccine according to claim 27, in combination with an anti-tumor agent selected from the group consisting of cyclophosphamide, carboplatin, paclitaxel and antibody-cytokine fusion protein NHS-IL12.
